(19) United States
(12) Patent Application Publication
(10) Pub. No.: US 2004/0204379 A1
(43) Pub. Date: Oct. 14, 2004

(54) Combination enzyme replacement, gene therapy and small molecule therapy for lysosomal storage diseases

(76) Inventors: Seng H. Cheng, Wellesley, MA (US); David Meeker, Concord, MA (US)

Correspondence Address: Finnegan, Henderson, Farabow,, Garrett & Dunner, L.L.P., 1300 I Street, N.W., Washington, DC, 20005-3315, US

(21) Appl. No.: 10/758,773

(22) Filed: Jan. 16, 2004

Related U.S. Application Data
(63) Continuation-in-part of application No. 09/884,526, filed on Jun. 19, 2001.
(60) Provisional application No. 60/212,377, filed on Jun. 19, 2000.

(51) Int. Cl. A61K 48/00

(52) U.S. Cl. 514/44

(57) ABSTRACT

This invention provides various combinations of enzyme replacement therapy, gene therapy, and small molecule therapy for the treatment of lysosomal storage diseases.

### DESCRIPTION OF THE INVENTION

This application is a continuation-in-part of U.S. patent application Ser. No. 09/884,526, filed Jun. 19, 2001, which claims priority to U.S. provisional application Serial No. 60/212,377 filed Jun. 19, 2000, both of which are incorporated-by-reference in their entirety.

### FIELD OF THE INVENTION

The present invention relates generally to the field of therapeutics for lysosomal storage diseases. More specifically, the invention relates to various combinations of enzyme replacement therapy, gene therapy, and small molecule therapy for the treatment of lysosomal storage diseases.

### BACKGROUND OF THE INVENTION

Each of the over thirty known lysosomal storage diseases (LSDs) is characterized by a similar pathogenesis, namely, a compromised lysosomal hydrolase. Generally, the activity of a single lysosomal hydrolytic enzyme is reduced or lacking altogether, usually due to inheritance of an autosomal recessive mutation. As a consequence, the substrate of the compromised enzyme accumulates undigested in lysosomes, producing severe disruption of cellular architecture and various disease manifestations.

A. Lysosomal Storage Diseases

Gaucher's disease, first described by Phillipe C. E. Gaucher in 1882, is the oldest and most common lysosomal storage disease known. Type 1 is the most common among three recognized clinical types and follows a chronic course which does not involve the nervous system. Types 2 and 3 both have a CNS component, the former being an acute infantile form with death by age two and the latter a subacute juvenile form. The incidence of Type 1 Gaucher's disease is about one in 50,000 live births generally and about one in 400 live births among Ashkenazim (see generally Kolodny et al., 1998, “Storage Diseases of the Reticuloendothelial System”, In: Nathan and Oski's Hematology of Infancy and Childhood, 5th ed., vol. 2, David G. Nathan and Stuart H. Orkin, Eds., W. B. Saunders Co., pages 1461-1507). Also known as glucosylceramide lipidosis, Gaucher's disease is caused by inactivation of the enzyme glucocerebrosidase and accumulation of glucocerebroside. Glucocerebrosidase normally catalyzes the hydrolysis of glucocerebroside to glucose and ceramide. In Gaucher's disease, glucocerebroside accumulates in tissue macrophages which become engorged and are typically found in liver, spleen and bone marrow and occasionally in lung, kidney and intestine. Secondary hematologic sequelae include severe anemia and thrombocytopenia in addition to the characteristic progressive hepatosplenomegaly and skeletal complications, including osteonecrosis and osteopenia with secondary pathological fractures.

Fabry disease is an X-linked recessive LSD characterized by a deficiency of α-galactosidase A (α-Gal A), also known as ceramide trihexosidase, which leads to vascular and other disease manifestations via accumulation of glycosphingolipids with terminal α-galactosyl residues, such as globotriaosylceramide (GL-3) (see generally Desnick R J et al., 1995, α-Galactosidase A Deficiency: Fabry Disease, In: The Metabolic and Molecular Bases of Inherited Disease, Scriver et al., eds., McGraw-Hill, N.Y., 7th ed., pages 2741-2784). Symptoms may include anhidrosis (absence of sweating), painful fingers, left ventricular hypertrophy, renal manifestations, and ischemic strokes. The severity of symptoms varies dramatically (Grewal R P, 1994, Stroke in Fabry's Disease, J. Neurol. 241, 153-156). A variant with manifestations limited to the heart is recognized, and its incidence may be more prevalent than once believed (Nakao S, 1995, An A typical Variant of Fabry's Disease in Men with Left Ventricular Hypertrophy, N. Engl. J. Med. 333, 288-293). Recognition of unusual variants can be delayed until quite late in life, although diagnosis in childhood is possible with clinical vigilance (Ko Y H et al., 1996, A typical Fabry's Disease—An Oligosymptomatic Variant, Arch. Pathol. Lab. Med. 120, 86-89; Mendez M F et al., 1997, The Vascular Dementia of Fabry's Disease, Dement. Geriatr. Cogn. Disord. 8, 252-257; Shelley E D et al., 1995, Painful Fingers, Heat Intolerance, and Telangiectases of the Ear: Easily Ignored Childhood Signs of Fabry Disease, Pediatric Derm. 12, 215-219). The mean age of diagnosis of Fabry disease is 29 years.

Niemann-Pick disease, also known as sphingomyelin lipidosis, comprises a group of disorders characterized by foam cell infiltration of the reticuloendothelial system. Foam cells in Niemann-Pick become engorged with sphingomyelin and, to a lesser extent, other membrane lipids including cholesterol. Niemann-Pick is caused by inactivation of the enzyme sphingomyelinase in Types A and B disease, with 27-fold more residual enzyme activity in Type B (see Kolodny et al., 1998, Id.). The pathophysiology of major organ systems in Niemann-Pick can be briefly summarized as follows. The spleen is the most extensively involved organ of Type A and B patients. The lungs are involved to a variable extent, and lung pathology in Type B patients is the major cause of mortality due to chronic bronchopneumonia. Liver involvement is variable, but severely affected patients may have life-threatening cirrhosis, portal hypertension, and ascites. The involvement of the lymph nodes is variable depending on the severity of disease. Central nervous system (CNS) involvement differentiates the major types of Niemann-Pick. While most Type B patients do not experience CNS involvement, it is characteristic in Type A patients. The kidneys are only moderately involved in Niemann Pick disease.

The mucopolysaccharidoses (MPS) comprise a group of LSDs caused by deficiency of enzymes which catalyze the degradation of specific glycosaminoglycans (mucopolysaccharides or GAGs) known as dermatan sulfate and heparan sulfate. GAGs contain long unbranched polysaccharides characterized by a repeating disaccharide unit and are found in the body linked to core proteins to form proteoglycans. Proteoglycans are located primarily in the extracellular matrix and on the surface of cells where they lubricate joints and contribute to structural integrity (see generally Neufeld et al., 1995, The Mucopolysaccharidoses, In: The Metabolic and Molecular Bases of Inherited Diseases, Scriver et al., eds., McGraw-Hill, N.Y., 7th ed., pages 2465-2494).

The several mucopolysaccharidoses are distinguished by the particular enzyme affected in GAG degradation. For example, MPS I (Hurler-Scheie) is caused by a deficiency of α-L-iduronidase which hydrolyzes the terminal α-L-iduronic acid residues of dermatan sulfate. Symptoms in MPS I vary along a clinical continuum from mild (MPS IS or Scheie disease) to intermediate (MPS IHS or Hurler-Scheie disease) to severe (MPS IH or Hurler disease), and the clinical presentation correlates with the degree of residual enzyme activity. The mean age at diagnosis for Hurler syndrome is about nine months, and the first presenting symptoms are often among the following: coarse facial features, skeletal abnormalities, clumsiness, stiffness, infections and hernias (Cleary M A and Wraith J E, 1995, The Presenting Features of Mucopolysaccharidosis Type III (Hurler Syndrome), Acta. Paediatr. 84, 337-339; Colville G A and Bax M A, 1996, Early Presentation in the Mucopolysaccharide Disorders, Child: Care, Health and Development 22, 31-36).

Other examples of mucopolysaccharidoses include Hunter (MPS II or iduronate sulfatase deficiency), Morquio (MPS IV; deficiency of galactosamine-6-sulfatase and b-galactosidase in types A and B, respectively) and Maroteaux-Lamy (MPS VI or arylsulfatase B deficiency) (see Neufeld et al., 1995, Id.; Kolodny et al., 1998, Id.).

Pompe disease (also known as glycogen storage disease type II, acid maltase deficiency and glycogenosis type II) is an autosomal recessive LSD characterized by a deficiency of α-glucosidase (also known as acid α-glucosidase and acid maltase). The enzyme α-glucosidase normally participates in the degradation of glycogen to glucose in lysosomes; it can also degrade maltose (see generally Hirschhorn R, 1995, Glycogen Storage Disease Type II: Acid α-Glucosidase (Acid Maltase) Deficiency, In: The Metabolic and Molecular Bases of Inherited Disease, Scriver et al., eds., McGraw-Hill, N.Y., 7th ed., pages 2443-2464). The three recognized clinical forms of Pompe disease (infantile, juvenile and adult) are correlated with the level of residual α-glucosidase activity (Reuser A J et al., 1995, Glycogenosis Type II (Acid Maltase Deficiency), Muscle & Nerve Supplement 3, S61-S69).

Infantile Pompe disease (type I or A) is most common and most severe, characterized by failure to thrive, generalized hypotonia, cardiac hypertrophy, and cardiorespiratory failure within the second year of life. Juvenile Pompe disease (type II or B) is intermediate in severity and is characterized by a predominance of muscular symptoms without cardiomegaly. Juvenile Pompe individuals usually die before reaching 20 years of age due to respiratory failure. Adult Pompe disease (type III or C) often presents as a slowly progressive myopathy in the teenage years or as late as the sixth decade (Felice K J et al., 1995, Clinical Variability in Adult-Onset Acid Maltase Deficiency: Report of Affected Sibs and Review of the Literature, Medicine 74, 131-135).

In Pompe, it has been shown that α-glucosidase is extensively modified post-translationally by glycosylation, phosphorylation, and proteolytic processing. Conversion of the 110 kilodalton (kDa) precursor to 76 and 70 kDa mature forms by proteolysis in the lysosome is required for optimum glycogen catalysis.

1. Therapies for Lysosomal Storage Diseases

Several approaches are being used or pursued for the treatment of LSDs, most of which focus on gene therapy or enzyme replacement therapy for use alone in disease management. Additionally, researchers have identified a number of small molecules for use alone in the management of LSDs. Other, disease-specific approaches, are also under consideration.

a. Gene Therapy

Replacement of the defective enzyme in a patient with Fabry Disease is considered feasible using a recombinant retrovirus carrying the cDNA encoding α-Galactosidase A to transfect skin fibroblasts obtained from Fabry patients (Medin et al., 1996, Correction in trans for Fabry Disease: Expression, Secretion, and Uptake of α-Galactosidase A in Patient Derived Cells Driven by a High-Titer Recombinant Retroviral Vector, Proc. Natl. Acad. Sci. USA 93, 7917).

Although both adenoviral and adeno-associated virus (AAV)-mediated gene transfer are reportedly capable of producing therapeutic levels of α-galactosidase A, these were only attained with relatively high doses of the recombinant viral vectors (Takahashi et al. 2002, Mol. Ther. 5:731; Ziegler et al. 2002, Hum Gene Ther. 13:935; Jung et al. Proc. Natl. Acad. Sci. USA 98:2676). Moreover, viral mediated expression in the Fabry mouse model induced the production of antibodies against the hydrolase that attenuated expression (Ziegler et al. 1999, Hum. Gene Ther. 10:1667; Park et al. 2003, Proc. Natl. Acad. Sci. USA 100:3450). This induction of neutralizing antibodies could also present additional complications in the form of hypersensitivity reactions and the formation of circulating immune complexes (Ponce et al. 1997, Blood 90:43; Desnick et al. 2003 Annals Int. Med. 138:338). The magnitude of the response will likely be dependent on the underlying gene mutation with null mutations being more problematic. Hence, the development of strategies that improve the potency of the gene transfer vector and that facilitate the induction of immuno-tolerance are warranted if gene therapy is to be considered for Fabry disease.

In vitro studies have also suggested that gene therapy may be feasible in Pompe disease. Vectors are being developed from both recombinant retrovirus and recombinant adenovirus (Zaretsky J Z et al., 1997, Retroviral Transfer of Acid α-Glucosidase cDNA to Enzyme-Deficient Myoblasts Results in Phenotypic Spread of the Genotypic Correction by Both Secretion and Fusion, Human Gene Therapy 8, 1555-1563; Pauly D F et al., 1998, Complete Correction of Acid α-Glucosidase Deficiency in Pompe Disease Fibroblasts in Vitro, and Lysosomally Targeted Expression in Neonatal Rat Cardiac and Skeletal Muscle, Gene Therapy 5, 473-480).

Additionally, transfer and expression of the normal α-L-iduronidase gene into autologous bone marrow by retroviral gene transfer has also been demonstrated in non-clinical studies of Hurler Syndrome (Fairbairn et al., 1996, Long-Term in vitro Correction of α-L-lduronidase Deficiency (Hurler Syndrome) in Human Bone Marrow, Proc. Natl. Acad. Sci. U.S.A. 93, 2025-2030).

b. Enzyme Replacement Therapy

Enzyme replacement therapy involves intravenous administration of an exogenously-produced natural or recombinant enzyme. Enzyme replacement therapy proof-of-principle has been established in a Hurler animal model (Shull R M et al., 1994, Enzyme Replacement in a Canine Model of Hurler Syndrome, Proc. Natl. Acad. Sci. USA 91, 12937-12941). Others have developed effective methods for cell culture expression of recombinant enzyme in sufficient quantities to be collected for therapeutic use (Kakkis E D et al., 1994, Overexpression of the Human Lysosomal Enzyme α-L-lduronidase in Chinese Hamster Ovary Cells, Prot. Express. Purif. 5, 225-232). However, one unsolved problem is the development of antibodies against the replacement enzyme after long term therapy (Kakkis E D et al., 1996, Long-Term and High-Dose Trials of Enzyme Replacement Therapy in the Canine Model of Mucopolysaccharidosis I, Biochem. Molec. Med. 58, 156-167).

The use of enzyme replacement therapy has also been investigated for patients with Pompe disease. However, effective enzyme replacement therapy requires the use of a precursor α-glucosidase molecule for correct targeting to lysosomes (Van Der Ploeg A T et al., 1987, Breakdown of Lysosomal Glycogen in Cultured Fibroblasts from Glycogenosis Type II Patients After Uptake of Acid α-Glucosidase, J. Neurolog. Sci. 79, 327-336; Van Der Ploeg, A T et al., 1991, Intravenous Administration of Phosphorylated Acid α-Glucosidase-Leads to Uptake of Enzyme in Heart and Skeletal Muscle of Mice, J. Clin. Invest. 87, 513-518; Van Der Ploeg A T et al., 1988, Prospect for Enzyme Replacement Therapy in Glycogenosis II Variants: A study on Cultured Muscle Cells, J. Neurol. 235, 392-396; Van Der Ploeg A T et al., 1988, Receptor-Mediated Uptake of Acid α-Glucosidase Corrects Lysosomal Glycogen Storage in Cultured Skeletal Muscle, Pediatr. Res. 24, 90-94). Despite the requirement for a robust production method for human recombinant α-glucosidase, animal and in vitro studies have provided reason for optimism (Van Hove J L K et al., 1996, High-Level Production of Recombination Human Lysosomal Acid α-Glucosidase in Chinese Hamster Ovary Cells Which Targets to Heart Muscle and Corrects Glycogen Accumulation in Fibroblasts from Patients with Pompe Disease, Proc. Natl. Acad. Sci. USA 93, 65-70; Kikuchi T et al., 1998, Clinical and Metabolic Correction of Pompe Disease by Enzyme Therapy in Acid Maltase-Deficient Quail, J. Clin. Invest. 101, 827-833).

The demonstration that α-galactosidase A secreted from genetically-modified cells can cross-correct affected bystander cells in vitro and in vivo provided the basis for the successful development of an enzyme replacement therapy for Fabry disease (see e.g. Neufeld et al. 1991, Annu Rev. Biochem. 60:257; loannou, et al. 2001, μm. J. Hum. Genet. 68:14; Eng et al. 2001, N. Engl. J. Med. 345:55; Schiffmann et al. 2001, JAMA 285:2743). Cross-correction is facilitated primarily by the mannose-6-phosphate receptor that is present ubiquitously on most cell types (Neufeld et al. 1991, Annu Rev. Biochem. 60:257). This ability of the secreted hydrolase to facilitate metabolic cooperativity also extends to enzyme that is generated by gene augmentation therapy (Medin et al. 1996, Correction in trans for Fabry Disease: Expression, Secretion, and Uptake of α-Galactosidase A in Patient Derived Cells Driven by a High-Titer Recombinant Retroviral Vector Proc. Natl. Acad. Sci. USA 93:7917). Adenoviral and AAV-mediated transductions of various depot organs have been shown to result in the secretion of α-galactosidase A with the appropriate recognition marker for mannose-6-phosphate receptor-mediated endocytosis.

C. Small Molecule Therapy

Recently, a variety of studies have been conducted using several small molecules for storage disease therapy. One class of molecules inhibits upstream generation of lysosomal hydrolase substrate to relieve the input burden to the defective enzyme. This approach has been dubbed “substrate deprivation” therapy. One example of this class of molecules is N-butyldeoxynojirimycin (NB-DNJ), an inhibitor of the ceramide-specific glucosyltransferase (i.e. glucosylceramide synthase) which catalyzes the first step in the synthesis of glycosphingolipids (GSLs). NB-DNJ has been tested in mouse models of Sandhoff disease (Jeyakumar et al., 1999, Proc. Natl. Acad. Sci. USA 96, 6388-6393), Tay-Sachs disease (Platt et al., 1997, Science 276, 428-431), as well as in humans with Gaucher's disease (Cox et al., 2000, Lancet 355, 1481-1485), resulting in an amelioration of symptoms in each of these diseases. A variety of deoxynojirimycin (DNJ) derivatives have also been synthesized as research tools intended for the selective inhibition of the non-lysosomal glucosylceramidase at concentrations in which glucosylceramide synthase and other enzymes are not affected (Overkleeft et al., 1998, J. Biol. Chem. 273, 26522-26527). Certain uses of glucosylceramide synthase inhibitors of the DNJ type either alone (WO 00/62780) or in combination with a glycolipid degrading enzyme (WO 00/62779) have been described.

Another example of the substrate deprivation class of molecules are the amino ceramide-like small molecules which have been developed for glucosylceramide synthase inhibition. Glucosylceramide synthase catalyzes the first glycosylation step in the synthesis of glucosylceramide-based glycosphingolipids. Glucosylceramide itself is the precursor of hundreds of different glycosphingolipids. Amino ceramide-like compounds have been developed for use in Fabry disease (Abe et al., 2000, J. Clin. Invest. 105, 1563-1571; Abe et al., 2000, Kidney Int'l 57, 446-454) and Gaucher's disease (Shayman et al., 2000, Meth. Enzymol. 31, 373-387; U.S. Pat. Nos. 5,916,911; 5,945,442; 5,952,370; 6,030,995; 6,040,332 and 6,051,598). A variety of amino ceramide-like analogues have been synthesized as improved inhibitors of glucosylceramide synthase (see e.g. Lee et al., 1999, J. Biol. Chem. 274, 14662-14669).

Aminoglycosides such as gentamicin and G418 are small molecules which promote read-through of premature stop-codon mutations. These so-called stop-mutation suppressors have been used in Hurler cells to restore a low level of α-L-iduronidase activity (Keeling et al., 2001, Hum. Molec. Genet. 10, 291-299). They have also been developed for use in treating cystic fibrosis individuals having stop mutations (U.S. Pat. No. 5,840,702). Small molecule chaperones or stabilizers of mutant lysosomal enzymes may also have utility (see e.g. Fan et al., 1999, Nat. Med. 5, 112-115 and Sawkar et al., 2002, Proc. Natl. Acad. Sci. USA 99, 15428-15433).

d. Other Therapies

Various other, disease-specific, treatments have been attempted. For example, a high protein diet in adult Pompe has been suggested to combat muscle wasting, but was effective in improving respiratory or muscle function in only 25% of individuals (Bodamer O A F et al., 1997, Dietary Treatment in Late-Onset Acid Maltase Deficiency, Eur. J. Pediatr. 156, S39-S42). In Hurler disease, bone marrow transplantation has shown limited benefits but carries significant risks (Guffon N et al., 1998, Follow-up of Nine Patients with Hurler Syndrome After Bone Marrow Transplantation, J. Pediatr. 133, 119-125; Gullingsrud E O et al., 1998, Ocular Abnormalities in the Mucopolysaccharidoses After Bone Marrow Transplantation, Ophthalmology 105, 1099-1105; Masterson E L et al., 1.996, Hip Dysplasia in Hurler's Syndrome: Orthopaedic Management After Bone Marrow Transplantation, J. Pediatric Orthopaedics 16, 731-733; Peters C et al., 1998, Hurler Syndrome: Past, Present and Future, J. Pediatr. 133, 7-9; Peters C et al., 1998, Hurler Syndrome: II. Outcome of HLA-Genotypically Identical Sibling and HLA-Haploidentical: Related Donor Bone Marrow Transplantation in Fifty-Four Children, Blood 91, 2601-2608). Early surgical intervention for nerve compression has been reported to improve hand function in individuals with Hurler disease (Van Heest A E et al., 1998, Surgical Treatment of Carpal Tunnel Syndrome and Trigger Digits in Children with Mucopolysaccharide Storage Disorders, J. Hand Surgery 23A, 236-243).

Kolodny et al. have provided a general overview of several approaches for treatment of LSDs in current use or development, including bone marrow transplantation, enzyme replacement therapy, and gene therapy (Kolodny et al., 1998, Id.). However, a need exists for defined and improved combination therapies that overcome significant limitations associated with each of these treatment modalities when used alone or with existing combination regimens. The present invention meets this need by providing improved approaches utilizing combinations of two or more of enzyme replacement therapy, gene therapy, where the expression of the transgene may be regulated by tissue specific regulatory elements, e.g., promoters and enhancers, and small molecule therapy.

### SUMMARY OF THE INVENTION

In certain embodiments, this invention provides various combinations of enzyme replacement therapy, gene therapy, and small molecule therapy for the treatment of lysosomal storage diseases. Several general approaches are provided. Each general approach involves combining at least two of enzyme replacement therapy (ERT), gene therapy (GT), and small molecule therapy (SMT) in a manner which optimizes clinical benefit, i.e., treatment, while minimizing disadvantages associated with using GT or ERT or SMT alone.

In certain embodiments the invention provides a method of treating a subject diagnosed as having a lysosomal storage disease comprising first administering a gene therapy vector encoding a lysosomal hydrolase under the control of a tissue specific promoter and then administering:

(a) an exogenously produced natural or recombinant lysosomal hydrolase;

(b) a small molecule capable of treating a lysosomal storage disease, or

(c) both (a) and (b), such that the lysosomal storage disease is treated.

In certain embodiments the invention provides a method of treating a subject diagnosed as having Fabry disease comprising first administering a gene therapy vector encoding α-galactosidase A under the control of a human albumin promoter and 2 copies of a human prothrombin enhancer and then administering:

(a) an exogenously produced natural or recombinant α-galactosidase A;

(b) a small molecule capable of treating Fabry disease, or

(c) both (a) and (b), such that the Fabry disease is treated.

In certain embodiments, the invention provides a composition useful for treating a lysosomal storage disease comprising a gene therapy vector encoding a lysosomal hydrolase under the control of a tissue specific promoter and optionally a tissue specific enhancer and: (a) an exogenously produced natural or recombinant lysosomal hydrolase; and (b) a small molecule capable of treating a lysosomal storage disease, or (c) both (a) and (b).

In certain embodiments, the invention provides a composition useful for treating Fabry disease comprising a gene therapy vector encoding α-galactosidase A under the control of a human albumin promoter and 2 copies of a human prothrombin enhancer and:

a) an exogenously produced natural or recombinant α-galactosidase A; or

b) a small molecule capable of treating Fabry disease or (c) both (a) and (b).

In certain embodiments, enzyme replacement therapy may be used as a de-bulking strategy (i.e. to initiate treatment), followed by or simultaneously supplemented with gene therapy and/or small molecule therapy. An advantage of ERT, whether used for de-bulking and/or for more long-term care, is the much broader clinical experience available to inform the practitioner's decisions. Moreover, a subject can be effectively titrated with ERT during the de-bulking phase by, for example, monitoring biochemical metabolites in urine or other body samples, or by measuring affected organ volume. A major disadvantage of traditional ERT is the frequency of the administration required, typically involving intravenous injection on a weekly or bi-weekly basis. Certain embodiments of the invention address this problem by providing for administration of a gene therapy vector encoding a lysosomal hydrolase under the control of a tissue specific promoter, e.g., a liver specific promoter. Use of a tissue specific promoter allows for the targeting of the gene therapy vector to a depot organ, e.g., the liver, which provides basal level expression of the trans gene, thus requiring less frequent and/or smaller doses of ERT.

In certain embodiments, gene therapy may also be administered as an effective method to de-bulk a subject, followed by or supplemented with enzyme replacement therapy and/or small molecule therapy as needed (e.g. when a gene therapy vector immune response precludes further immediate gene therapy, or when a gene therapy vector is administered in low dose to avoid an immune response, and consequently needs supplementation to provide therapeutic enzyme amounts). The major advantage of gene therapy is the prolonged time course of effective treatment which can be achieved. The persistence of the transduced gene is such that therapeutically beneficial enzyme is produced for a duration of from several months to as long as one to several years, or even indefinitely, following a single administration of the gene therapy vector. This low frequency of administration is in stark contrast to enzyme replacement therapy, wherein a recombinantly-produced protein is generally required to be administered on at least a weekly or bi-weekly schedule.

Alternating among GT and ERT and SMT, or supplementing low-dose GT with ERT and/or SMT, provides a strategy for simultaneously taking advantage of the strengths and addressing the weaknesses associated with each therapy employed alone. On one hand, a vector immune response in a subject undergoing gene therapy can be successfully addressed by switching the subject to enzyme replacement therapy until the vector immune response subsides. On the other hand, a subject currently undergoing, for example, a bi-weekly enzyme replacement therapy dosing regimen can be offered an “ERT holiday” (e.g. using a GT administration which is effective for six months or longer, alone or in combination with SMT) wherein frequent enzyme injections are not required therapy.

Accordingly, in certain embodiments, this invention provides a method of combination therapy for treatment of a subject diagnosed as having a lysosomal storage disease comprising: (a) monitoring the subject for an immune response to a gene therapy; and (b) treating the subject with an enzyme replacement therapy prior to or when the immune response to the gene therapy reaches a parameter determined to be clinically unacceptable.

In certain embodiments, this invention provides a method of combination therapy for treatment of a subject diagnosed as having a lysosomal storage disease comprising: (a) monitoring the subject for an immune response to a gene therapy; and (b) treating the subject with a small molecule therapy prior to or when the immune response to the gene therapy reaches a parameter determined to be clinically unacceptable.

In certain embodiments, this invention provides a method of combination therapy for treatment of a subject diagnosed as having a lysosomal storage disease comprising: (a) administering a low dose gene therapy to the subject; (b) monitoring the subject for a disease status indicator in response to the low dose gene therapy; and (c) administering a supplemental enzyme replacement therapy prior to or when the disease status indicator reaches a parameter determined to be clinically unacceptable.

In certain embodiments, this invention provides a method of combination therapy for treatment of a subject diagnosed as having a lysosomal storage disease comprising: (a) administering a low dose gene therapy to the subject; (b) monitoring the subject for a disease status indicator in response to the low dose gene therapy; and (c) administering a supplemental small molecule therapy prior to or when the disease status indicator reaches a parameter determined to be clinically unacceptable.

In certain embodiments, this invention provides a method of combination therapy for treatment of a subject diagnosed as having a lysosomal storage disease comprising: (a) administering a low dose gene therapy to the subject; (b) monitoring the subject for a disease status indicator in response to the low dose gene therapy; and (c) simultaneously administering a supplemental enzyme replacement therapy and a small molecule therapy prior to or when the disease status indicator reaches a parameter determined to be clinically unacceptable.

In certain embodiments, this invention provides a method of combination therapy for treatment of a subject diagnosed as having a lysosomal storage disease comprising: (a) administering a low dose gene therapy to the subject; (b) monitoring the subject for a disease status indicator in response to the low dose gene therapy; and (c) alternating between a supplemental enzyme replacement therapy and a small molecule therapy prior to or when the disease status indicator reaches a parameter determined to be clinically unacceptable.

In certain embodiments, this invention provides a method of combination therapy for treatment of a subject diagnosed as having a lysosomal storage disease comprising alternating between administration of an enzyme replacement therapy and a gene therapy.

In certain embodiments, this invention provides a method of combination therapy for treatment of a subject diagnosed as having a lysosomal storage disease comprising alternating between administration of an enzyme replacement therapy and a small molecule therapy.

In certain embodiments, this invention provides a method of combination therapy for treatment of a subject diagnosed as having a lysosomal storage disease comprising alternating between administration of a gene therapy and a small molecule therapy.

In certain embodiments, this invention provides a method of combination therapy for treatment of a subject diagnosed as having a lysosomal storage disease comprising alternating between administration of an enzyme replacement therapy, a gene therapy, and a small molecule therapy.

In certain embodiments, this invention provides a method of combination therapy for treatment of a subject diagnosed as having a lysosomal storage disease comprising alternating between administration of an enzyme replacement therapy, said enzyme replacement therapy being simultaneously administered with a small molecule therapy, and a gene therapy.

In certain embodiments, this invention provides a method of combination therapy for treatment of a subject diagnosed as having a lysosomal storage disease comprising alternating between administration of an enzyme replacement therapy and a gene therapy, said gene therapy being simultaneously administered with a small molecule therapy.

In certain embodiments, this invention provides a method of combination therapy for treatment of a subject diagnosed as having a lysosomal storage disease comprising alternating between administration of a small molecule therapy and a gene therapy, said gene therapy being simultaneously administered with an enzyme replacement therapy.

In certain embodiments, this invention provides a method of combination therapy for treatment of a subject diagnosed as having a lysosomal storage disease comprising alternating between administration of a gene therapy and an enzyme replacement therapy, wherein each of said gene therapy and said enzyme replacement therapy is simultaneously administered with a small molecule therapy.

In certain embodiments, this invention provides a method of combination therapy for treatment of a subject diagnosed as having a lysosomal storage disease comprising alternating between administration of a gene therapy and a small molecule therapy, wherein each of said gene therapy and said small molecule therapy is simultaneously administered with an enzyme replacement therapy.

In certain embodiments, this invention provides a method of combination therapy for treatment of a subject diagnosed as having a lysosomal storage disease comprising alternating between administration of an enzyme replacement therapy and a small molecule therapy, wherein each of said enzyme replacement therapy and said small molecule therapy is simultaneously administered with a gene therapy.

In certain embodiments, this invention provides a method of combination therapy for treatment of a subject diagnosed as having a lysosomal storage disease comprising simultaneously administering a gene therapy and an enzyme replacement therapy.

In certain embodiments, this invention provides a method of combination therapy for treatment of a subject diagnosed as having a lysosomal storage disease comprising simultaneously administering a gene therapy and a small molecule therapy.

In certain embodiments, this invention provides a method of combination therapy for treatment of a subject diagnosed as having a lysosomal storage disease comprising simultaneously administering an enzyme replacement therapy and a small molecule therapy.

In certain embodiments, this invention provides a method of combination therapy for treatment of a subject diagnosed as having a lysosomal storage disease comprising simultaneously administering a gene therapy, an enzyme replacement therapy and a small molecule therapy.

In certain embodiments, this invention provides a method of combination therapy for treatment of a subject diagnosed as having a lysosomal storage disease comprising: (a) administering an enzyme replacement therapy for a period of at least six months to de-bulk the subject; and (b) administering a gene therapy to the de-bulked subject in order to provide an infusion vacation for a period of at least six months.

In certain embodiments, this invention provides a method for determining when to substitute repeated administration of gene therapy with enzyme replacement therapy in the treatment of Gaucher's disease in a subject comprising: (a) monitoring an immune status indicator in the subject; (b) administering enzyme replacement therapy in lieu of repeated administration of gene therapy prior to or when the immune status indicator reaches a value determined to be clinically unacceptable. In one embodiment, the enzyme replacement therapy administered in step (b) comprises a dosage regimen of from 2.5 U/kg three times a week to 60 U/kg once every two weeks.

In certain embodiments, this invention provides a method for determining when to substitute repeated administration of gene therapy with small molecule therapy in the treatment of Gaucher's disease in a subject comprising: (a) monitoring an immune status indicator in the subject; (b) administering small molecule therapy in lieu of repeated administration of gene therapy prior to or when the immune status indicator reaches a value determined to be clinically unacceptable.

In certain embodiments, this invention provides a method for determining when to substitute repeated administration of gene therapy with enzyme replacement therapy in the treatment of Gaucher's disease in a subject comprising:

(a) monitoring an immune status indicator in the subject; (b) administering a combination of enzyme replacement therapy and small molecule therapy in lieu of repeated administration of gene therapy prior to or when the immune status indicator reaches a value determined to be clinically unacceptable. In one embodiment, the enzyme replacement therapy administered in step (b) comprises a dosage regimen of from 2.5 U/kg three times a week to 60 U/kg once every two weeks.

In certain embodiments, this invention provides a method for determining when to substitute repeated administration of gene therapy with enzyme replacement therapy in the treatment of Fabry's disease in a subject comprising: (a) monitoring globotriaosylceramide and pain in the subject; (b) administering enzyme replacement therapy instead of repeated administration of gene therapy prior to or when globotriaosylceramide or pain reaches a value determined to be clinically unacceptable.

In certain embodiments, this invention provides a method for determining when to substitute repeated administration of gene therapy with small molecule therapy in the treatment of Fabry's disease in a subject comprising: (a) monitoring globotriaosylceramide and pain in the subject; (b) administering small molecule therapy instead of repeated administration of gene therapy prior to or when globotriaosylceramide or pain reaches a value determined to be clinically unacceptable.

In certain embodiments, this invention provides a method for determining when to substitute repeated administration of gene therapy with small molecule therapy in the treatment of Fabry's disease in a subject comprising: (a) monitoring globotriaosylceramide and pain in the subject; (b) administering a combination of small molecule therapy and enzyme replacement therapy instead of repeated administration of gene therapy prior to or when globotriaosylceramide or pain reaches a value determined to be clinically unacceptable.

In certain embodiments, this invention provides a method for determining when to substitute repeated administration of gene therapy with enzyme replacement therapy in the treatment of Fabry's disease in a subject comprising: (a) monitoring globotriaosylceramide and pain in the subject; (b) administering enzyme replacement therapy instead of repeated administration of gene therapy prior to or when globotriaosylceramide and pain reach values determined to be clinically unacceptable.

In certain embodiments, this invention provides a method for determining when to substitute repeated administration of gene therapy with small molecule therapy in the treatment of Fabry's disease in a subject comprising: (a) monitoring globotriaosylceramide and pain in the subject; (b) administering small molecule therapy instead of repeated administration of gene therapy prior to or when globotriaosylceramide and pain reach values determined to be clinically unacceptable.

In certain embodiments, this invention provides a method for determining when to substitute repeated administration of gene therapy with small molecule therapy in the treatment of Fabry's disease in a subject comprising: (a) monitoring globotriaosylceramide and pain in the subject; (b) administering a combination of small molecule therapy and enzyme replacement therapy instead of repeated administration of gene therapy prior to or when globotriaosylceramide and pain reach values determined to be clinically unacceptable.

In the various combination therapies of the invention, it will be understood that administering small molecule therapy may occur prior to, concurrently with, or after, administration of one or more of the other therapies. Similarly, administering enzyme replacement therapy may occur prior to, concurrently with, or after, administration of one or more of the other therapies. Finally, administering gene therapy may occur prior to; concurrently With, or after, administration of one or more of the other therapies.

In any of the foregoing embodiments of the invention, the lysosomal storage disease is selected from the group consisting of Gaucher, Niemann-Pick, Farber, G_{M1}-gangliosidosis, G_{M2}-gangliosidosis (Sandhoff), Tay-Sachs, Krabbe, Hurler-Scheie (MPS I), Hunter (MPS II), Sanfilippo (MPS III) Type A, Sanfilippo (MPS ll) Type B, Sanfilippo (MPS ll) Type C, Sanfilippo (MPS III) Type D, Marquio (MPS IV) Type A, Marquio (MPS IV) Type B, Maroteaux-Lamy (MPS VI), Sly (MPS VII), mucosulfatidosis, sialidoses, mucolipidosis II, mucolipidosis III, mucolipidosis IV, Fabry, Schindler, Pompe, sialic acid storage disease, fucosidosis, mannosidosis, aspartylglucosaminuria, Wolman, and neuronal ceroid lipofucsinoses.

Further, in certain embodiments, the foregoing combination therapies provide an effective amount of at least one enzyme selected from the group consisting of glucocerebrosidase, sphingomyelinase, ceramidase, G_{M1}-ganglioside-β-galactosidase, hexosaminidase A, hexosamimidase B, β-galactocerebrosidase, α-L-iduronidase, iduronate sulfatase, heparan-N-sulfatase, N-acetyl-α-glucosaminidase, acetyl CoA:α-glucosaminide acetyl-transferase, N-acetyl-α-glucosamine-6-sulfatase, galactosamine-6-sulfatase, β-galactosidase, galactosamine-4-sulfatase (arylsulfatase B), β-glucuronidase, arylsulfatase A, arylsulfatase C, α-neuraminidase, N-acetyl-glucosamine-1-phosphate transferase, α-galactosidase A, α N acetylgalactosaminidase, α-glucosidase, α-fucosidase, α-mannosidase, aspartylglucosamine amidase, acid lipase, and palmitoyl-protein thioesterase (CLN-1).

Still further, in certain embodiments, the foregoing combination therapy produces a diminution in at least one stored material selected from the group consisting of glucocerebroside, sphingomyelin, ceramide, G_{M1}-ganglioside, G_{M2}-ganglioside, globoside, galactosylceramide, dermatan sulfate, heparan sulfate, keratan sulfate, sulfatides, mucopolysaccharides, sialyloligosaccharides, glycoproteins, sialyloligosaccharides, glycolipids, globotriaosylceramide, O-linked glycopeptides, glycogen, free sialic acid, fucoglycolipids, fucosyloligosaccharides, mannosyloligosaccharides, aspartylglucosam ine, cholesteryl esters, triglycerides, and ceroid lipofuscin pigments.

In one embodiment of the invention, the small molecule therapy comprises administering to the subject an effective amount of deoxynojirimycin or a deoxynojirimycin derivative. In another embodiment, the deoxynojirimycin derivative is N-propyldeoxynojirimycin, N-butyldeoxynojirimycin, N-butyldeoxygalactonojirimycin, N-pentlydeoxynojirimycin, N-heptyldeoxynojirimycin, N-pentanoyldeoxynojirimycin, N-(5-adamantane-1-ylmethoxy)pentyl)-deoxynojirimycin, N-(5-cholesteroxypentyl)-deoxynojirimycin, N-(4-adamantanemethanylcarboxy-1-oxo)-deoxynojirimycin, N-(4-adamantanylcarboxy-1-oxo)-deoxynojirimycin, N-(4-phenantrylcarboxy-1-oxo)-deoxynojirimycin, N-(4-cholesterylcarboxy-1-oxo)-deoxynojirimycin, or N-(4-b-cholestanylcarboxy-1-oxo)-deoxynojirimycin.

In other embodiments, the small molecule therapy comprises administering to the subject an effective amount of D-threo-1-phenyl-2-palmitoylamino-3-pyrrolidino-1-propanol (P4) or a P4 derivative. In another embodiment, the P4 derivative is selected from the group consisting of D-threo-4′-hydroxy-1-phenyl-2-palmitoylamino-3-pyrrolidino-1-propanol (4′-hydroxy-α4), D-threo-1-(3′,4′-trimethylenedioxy)phenyl-2-palmitoylamino-3-pyrrolidino-1-propanol (trimethylenedioxy-P4), D-threo-1-(3′,4′-methylenedioxy)phenyl-2-palmitoylamino-3-pyrrolidino-1-propanol (methylenedioxy-P4) and D-threo-1-(3′,4′-ethylenedioxy)phenyl-2-palmitoylamino-3-pyrrolidino-1-propanol (ethylenedioxy-P4 or D-t-et-P4).

In one embodiment of the invention, the lysosomal storage disease is attributable at least in part to a stop codon mutation in a gene encoding a lysosomal storage enzyme, and wherein the small molecule therapy comprises administering to the subject an effective amount of an aminoglycoside. In another embodiment, the aminoglycoside is gentamicin, G418, hygromycin B, paromomycin, tobramycin or Lividomycin A.

In other embodiments, the immune response to gene therapy is monitored by assay of an immune status indicator selected from the group consisting an antibody and a cytokine. In another embodiment, the cytokine is selected from the group consisting of IL-1α, IL-2, IL-4, IL-8, IL-10, G-CSF, GM-CSF, M-CSF, α-interferon, β-interferon and γ-interferon. In another embodiment, the antibody is specifically reactive with an antigen selected from the group consisting of a viral antigen, a lipid antigen and a DNA antigen.

In yet other embodiments, the lysosomal storage disease has at least one central nervous system manifestation and the small molecule therapy comprises AMP-DNJ.

In various embodiments of the invention, the subject may be a human or a non-human animal.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE DRAWINGS

FIG. 1 demonstrates the in vivo efficacy of combination enzyme replacement therapy plus small molecule therapy in Fabry disease. FIG. 1A shows the study protocol for sequential combination of enzyme (α-galactosidase A) replacement followed by small molecule administration (NB-DNJ, AMP-DNJ or D-t-et-P4) on globotriaosylceramide (GL3) re-accumulation in Fabry mice. FIG. 1B shows the results of study protocol for Fabry mouse liver tissue. GL3 re-accumulation at four weeks (μg GL3 per μm liver tissue) is plotted on the ordinate versus absence of small molecule treatment (Vehicle) or daily intra-peritoneal small molecule therapy with D-t-et-P4 (at either 5 mg/kg or 0.5 mg/kg), NB-DNJ (at 500 mg/kg), or AMP-DNJ (at 100 mg/kg). Baseline GL3 level in Fabry mouse liver (about 0.1 μg/gm liver tissue) shows the GL3 level achieved at two weeks following a single α-galactosidase A intravenous infusion. In control animals receiving daily Vehicle administration, GL3 re-accumulated to about 0.8 μg/gm liver tissue at the four week time point. In marked contrast, D-t-et-P4 (5 mg/kg) and AMP-DNJ (100 mg/kg) reduced GL3 re-accumulation in Fabry mouse liver tissue to less than about 0.4 μg/gm or 0.3 μg/gm, respectively, at the four week time point.

FIG. 2 demonstrates the efficacy of administration of AAV2/CAAVHI-αgal into Fabry mice. Seven month old immune-suppressed Fabry mice were administered 5×10¹¹ particles of AAV2/CAAVHI-αgal via the tail vein. Animals were killed at 1, 2 and 3 months post-treatment and their organs analyzed for the levels of α-galactosidase A (A) and GL3 (B). An ELISA specific for human α-galactosidase A was used to measure the enzyme levels in the different tissue homogenates. The shaded area within the graph represents the range of α-galactosidase A levels observed in normal (C57BL6) mouse tissues. To measure the GL-3 levels, an ELISA based on the affinity of E. coli verotoxin to bind the glycosphingolipid was used. Data is expressed as mean±SEM (n=4 animals/time point).

FIG. 3 demostrates expression of α-galactosidase A from a CAAV and liver-restricted promoter. Six week old male BALB/c mice were injected intravenously with vehicle or with 3×10¹ particles of either AAV2/CAAVHI-αgal or AAV2/DC 190-αgal. Animals were killed at 1, 2 and 3 months post-injection and various organs collected for analysis. Blood was also collected by eyebleed at the same time intervals. An ELISA specific for human α-galactosidase A was used to detect protein levels in the different tissue homogenates and serum samples. The shaded area within the graph represents the range of α-galactosidase A levels observed in normal (C57BL/6) mouse tissues. Data are expressed as mean±SEM (n=5 animals/time point).

FIG. 4 demonstrates the longevity of expression of α-galactosidase A from the liver-restricted promoter. Six week old male BALB/c mice were injected intravenously with 3×10¹¹ particles of AAV2/DC190-αgal. Blood was collected periodically by eyebleed starting at day 30 until day 340 post-treatment. An ELISA specific for human α-galactosidase A was used to detect protein levels in the serum samples. Data is expressed as mean SEM (n=5 animals/time point).

FIG. 5 shows the quantitation of viral DNA and α-galactosidase A mRNA in various tissues following intravenous administration of the recombinant AAV vectors. Six week old male BALB/c mice were injected intravenously with 3×10¹¹ particles of either AAV2/CAAVHI-αgal or AAV2/DC190-αgal. Animals were killed 30 days later; their organs harvested and then assayed for the presence of AAV genomes (A) and human α-galactosidase A mRNA (B). The shaded areas within the graphs represent values that are below the range of reliable quantitation. Data are expressed as mean SEM (n=5 animals).

FIG. 6 shows the titer of antibodies to human α-galactosidase A and AAV2 in BALB/c mice. Groups of 4 male BALB/c mice were injected intravenously with vehicle or 3×10¹¹ particles of either AAV2/CAAVHI-αgal or AAV2/DC190-αgal. Mice were bled prior to treatment and subsequently on month 1, 2 and 3 post-treatment. Antibodies to human α-galactosidase A (A) and AAV2 (B) were quantitated using an ELISA as described in Example 4. Data is expressed as mean±SEM (n=5 animals/time point).

FIG. 7 shows the serum levels of α-galactosidase A and antibodies to α-galactosidase A following administration of AAV2/DC190-αgal and subsequent challenge with purified recombinant enzyme. Groups of 4 male BALB/c mice were administered increasing amounts of AAV2/DC190-αgal via the tail vein. Mice were bled 88 days later and the serum levels of α-galactosidase A quantitated by ELISA (A). At 6 months post-treatment, the mice were challenged intraperitoneally with 50 μg of purified recombinant human α-galactosidase A that had been emulsified in complete Freund's adjuvant. The animals were bled 38 days following the challenge and the serum levels of anti-α-galactosidase A antibodies determined (B). The open circles represent individual animals and the closed circles the mean of all the values. No PreRx refers to the group of mice that had not been treated with AAV2/DC190-αgal.

FIG. 8 demonstrates the efficacy of administration of AAV2/DC190-αgal into Fabry mice. Four month old male Fabry mice were injected intravenously with 5×10¹¹ particles of AAV2-DC190-αgal via the tail vein. Animals were killed at 1, 2 and 3 months post-treatment and their organs analyzed for the levels of α-galactosidase A (A) and GL3 (B). An ELISA specific for human α-galactosidase A was used to measure the enzyme levels in the different tissue homogenates. The shaded area within the graph represents the range of α-galactosidase A levels observed in normal (C57BU6) mouse tissues. To measure the GL-3 levels, an ELISA that was based on the affinity of E. coli verotoxin to bind the glycosphingolipid was used. Data are expressed as mean±SEM (n=4 animals/time point).

### DESCRIPTION OF THE EMBODIMENTS

The therapeutic methods of the invention described herein provide treatment options for the practitioner faced with management of various lysosomal storage diseases, as described in detail below. More specifically, the invention relates to various combinations of enzyme replacement therapy and gene therapy for the treatment of lysosomal storage diseases.

A partial list of known lysosomal storage diseases that can be treated in accordance with the invention is set forth in Table 1, including common disease name, material stored, and corresponding enzyme deficiency (adapted from Table 384 of Kolodny et al., 1998).

TABLE 1
Lysosomal Storage Diseases

| Disease | Material Stored | Enzyme Deficiency |
|---|---|---|
| Sphingolipidoses | | |
| Gaucher | Glucocerebroside | Glucocerebrosidase |
| Niemann-Pick | Sphingomyelin | Sphingomyelinase |
| Farber | Ceramide | Ceramidase |
| G_{M1}-gangliosidosis | G_{M1}-ganglioside, | G_{M1}-ganglioside-β- |
| | glycoprotein | galactosidase |
| G_{M2}-gangliosidosis | G_{M2}-ganglioside, | Hexosaminidase A |
| (Sandhoff) | globoside | and B |
| Tay-Sachs | G_{M2}-ganglioside | Hexosaminidase A |
| Krabbe | Galactosylceramide | β-Galacto- |
| | | cerebrosidase |
| Mucopolysaccharidoses | | |
| Hurler-Scheie (MPS I) | Dermatan sulfate, | α-L-iduronidase |
| | heparan sulfate | |
| Hunter (MPS II) | Dermatan sulfate, | Iduronate sulfatase |
| | heparan sulfate | |
| Sanfilippo (MPS III) | | |
| Type A | Heparan sulfate | Heparan-N-sulfatase |
| Type B | Heparan sulfate | N-acetyl-α- |
| | | glucosaminidase |
| Type C | Heparan sulfate | Acetyl CoA: |
| | | α-glucosaminide |
| | | acetyl-transferase |
| Type D | Heparan sulfate | N-acetyl-α-glucosa- |
| | | mine-6-sulfatase |
| Marquio (MPS IV) | | |
| Type A | Keratan sulfate | Galactosamine-6- |
| | | sulfatase |
| Type B | Keratan sulfate | β-galactosidase |
| Maroteaux-Lamy (MPS VI) | Dermatan sulfate | Galactosamine-4- |
| | | sulfatase |
| | | (arylsulfatase B) |
| Sly (MPS VII) | Dermatan sulfate, | β-glucuronidase |
| | heparan sulfate | |
| Mucosulfatidosis | Sulfatides, | Arylsulfatase A, B |
| | mucopolysaccharides | and C, |
| | | other sulfatases |
| Mucolipidoses | | |
| Sialidoses | Sialyloligosaccharides, | β-neuraminidase |
| | glycoproteins | |
| Mucolipidosis II | Sialyloligosaccharides, | High serum, low |
| | glycoproteins, | fibroblast enzymes; |
| | glycolipids | N-acetyl-glucos- |
| | | amine-1-phosphate |
| Mucolipidosis III | Glycoproteins, | transferase |
| | glycolipids | |
| Mucolipidosis IV | Glycolipids, | Same as above |
| | glycoproteins | Unknown |
| Other Diseases of | | |
| Complex | | |
| Carbohydrate Metabolism | | |
| Fabry | Globotriaosylceramide | α-gaIactosidase A |
| Schindler | O-linked glycopeptides | α-N-acetylgalactos- |
| Pompe | Glycogen | aminidase |
| Sialic acid storage disease | Free sialic acid | α-glucosidase |
| Fucosidosis | Fucoglycolipids, | Unknown |
| | fucosyloligo- | α-fucosidase |
| | saccharides | |
| Mannosidosis | Mannosyloligo- | |
| | saccharides | |
| Aspartylglucosaminuria | Aspartylgiucosamine | α-mannosidase |
| | | Aspartylglucos- |
| | | amine |
| Wolman | Cholesteryl esters, | amidase |
| | triglycerides | |
| Neuronal ceroid | Ceroid lipofuscin | |
| lipofucsinoses | pigments | Acid lipase |
| | | Palmitoyl-protein |
| | | thioesterase |
| | | (CLN-1) |

An “effective amount” of an enzyme, small molecule, or gene therapy, when delivered to a subject in a combination therapy of the invention, is an amount sufficient to improve the clinical course of a lysosomal storage disease, where clinical improvement is measured by any of the variety of defined parameters well known to the skilled artisan.

Any method known to the skilled artisan may be used to monitor disease status and the effectiveness of a combination therapy of the invention. Clinical monitors of disease status may include but are not limited to organ volume (e.g. liver, spleen), hemoglobin, erythrocyte count, hematocrit, thrombocytopenia, cachexia (wasting), and plasma chitinase levels (e.g. chitotriosidase). Chitotriosidase, an enzyme of the chitinase family, is known to be produced by macrophages in high levels in subjects with lysosomal storage diseases (see Guo et al., 1995, J. Inherit. Metab. Dis. 18, 717-722; den Tandt et al., 1996, J. Inherit. Metab. Dis. 19, 344-350; Dodelson de Kremer et al., 1997, Medicina (Buenos Aires) 57, 677-684; Czartoryska et al., 2000, Clin. Biochem. 33, 147 149; Czartoryska et al., 1998, Clin. Biochem. 31, 417-420; Mistry et al., 1997, Baillieres Clin. Haematol. 10, 817-838; Young et al., 1997, J. Inherit. Metab. Dis. 20, 595-602; Hollak et al., 1994, J. Clin. Invest. 93, 1288-1292).

Methods and formulations for administering the combination therapies of the invention include all methods and formulations well known in the art (see e.g. Remington's Pharmaceutical Sciences, 1980 and subsequent years, 16th ed. and subsequent editions, A. Oslo editor, Easton Pa.; Controlled Drug Delivery, 1987, 2nd rev., Joseph R. Robinson & Vincent H. L. Lee, eds., Marcel Dekker, ISBN: 0824775880; Encyclopedia of Controlled Drug Delivery, 1999, Edith Mathiowitz, John Wiley & Sons, ISBN: 0471148288; U.S. Pat. No. 6,066,626 and references cited therein; see also, references cited in sections below).

According to certain embodiments of the invention, the following general approaches are provided for combination therapy in the treatment of lysosomal storage diseases. Each general approach involves combining enzyme replacement therapy with gene therapy and/or with small molecule therapy in a manner consistent with optimizing clinical benefit while minimizing disadvantages associated with using each therapy alone.

In a first general approach to a combination therapy of the invention, enzyme replacement therapy (alone or in combination with small molecule therapy) is administered to initiate treatment (i.e. to de-bulk the subject), and gene therapy (alone or in combination with small molecule therapy) is administered after the de-bulking phase to achieve and maintain a stable, long-term therapeutic effect without the need for frequent intravenous ERT injections. For example, enzyme replacement therapy may be administered intravenously (e.g. over a one to two hour period) on a weekly or bi-weekly basis for one to several weeks or months, or longer (e.g. until an involved indicator organ such as spleen or liver shows a decrease in size). Moreover, the ERT phase of initial de-bulking treatment can be performed alone or in combination with a small molecule therapy. After this initial phase, gene therapy may be administered to achieve a prolonged clinical benefit that does not require frequent intravenous intervention. Depending on the nature of the gene therapy vector introduced, the gene therapy component of a combination therapy of the invention optimally will not need supplement for a period of six months, one year, or even indefinitely. An SMT component of a combination therapy can be adjusted as needed throughout the course of the storage disease by the skilled practitioner by monitoring well known clinical signs of disease progression or remission. A small molecule therapeutic component may be used where the small molecule is compatible with oral administration, thus providing further relief from frequent intravenous intervention.

In a second general approach to a combination therapy of the invention, gene therapy can be administered to de-bulk the subject, followed by or simultaneously supplemented with enzyme replacement therapy and/or small molecule therapy. Such an approach is particularly indicated where a lysosomal storage disease exhibits clinical pathology in an organ having a relatively low circulation (e.g. lymph nodes). In this scenario, deposition and long-term residence of the therapeutic gene by GT at a low-circulation site reduces the dependence of clinical success on repeated IV injections that may have trouble reaching the site. Enzyme replacement therapy and/or small molecule therapy is then used as needed to supplement or maintain the clinical benefit from gene therapy. Moreover, a relatively low dose of gene therapy may be initially employed, e.g., to minimize a vector immune response, supplemented with simultaneous enzyme replacement and/or small molecule therapy as needed to achieve the desired clinical result.

A third general approach to a combination therapy of the invention involves alternative dosing. In one embodiment of alternative dosing, enzyme replacement therapy and/or small molecule therapy may be administered during a period of time required for immune system recovery from an immune response raised against a gene therapy vector. In another embodiment of alternative dosing, gene therapy is administered to provide a prolonged period of time (e.g. six months to one year or longer) wherein weekly or bi-weekly intravenous enzyme infusions are not required (i.e. an “infusion vacation”). Of course, the GT component and the ERT component can each be supplemented with small molecule therapy as needed.

A variety of gene therapy vectors are available for the treatment of the various LSDs (described in detail below). For example, in vivo and ex vivo approaches to gene therapy may be implemented using viral or non-viral vectors. The central nervous system (CNS) is generally much harder to target than the reticuloendothelial system (RES) because of the blood-brain barrier (BBB). However, bone marrow cells transduced to express a therapeutic gene may provide some CNS benefit. Finally, cationic-lipid-plus-plasmid combinations are especially indicated for diseases that have lung involvement since they can, for example, be administered by aerosol at the disease locus.

Gene therapy and enzyme replacement therapy can provoke unwanted immune responses. Accordingly, in some embodiments, immunosuppressant agents may be used together with a gene therapy component or an enzyme replacement therapy component of a combination therapy of the invention. Such agents may also be used with a small molecule therapy component, but the need for intervention here is generally less likely. Any immunosuppressant agent known to the skilled artisan may be employed together with a combination therapy of the invention. Such immunosuppressant agents include but are not limited to cyclosporine, FK506, rapamycin, CTLA4-Ig, and anti-TNF agents such as etanercept (see e.g. Moder, 2000, Ann. Allergy Asthma Immunol. 84, 280-284; Nevins, 2000, Curr. Opin. Pediatr. 12, 146-150; Kurlberg et al., 2000, Scand. J. Immunol. 51, 224-230; Ideguchi et al., 2000, Neuroscience 95, 217-226; Potter et al., 1999, Ann. N.Y. Acad. Sci. 875, 159-174; Slavik et al., 1999, Immunol. Res. 19, 1-24; Gaziev et al., 1999, Bone Marrow Transplant. 25, 689-696; Henry, 1999, Clin. Transplant. 13, 209-220; Gummert et al., 1999, J. Am. Soc. Nephrol. 10, 1366-1380; Qi et al., 2000, Transplantation 69, 1275-1283). The anti-IL2 receptor (α-subunit) antibody daclizumab (e.g. ZenapaxJ), which has been demonstrated effective in transplant patients, can also be used as an immunosuppressant agent (see e.g. Wiseman et al., 1999, Drugs 58, 1029-1042; Beniaminovitz et al., 2000, N. Engl J. Med. 342, 613-619; Ponticelli et al., 1999, Drugs R. D. 1, 55-60; Berard et al., 1999, Pharmacotherapy 19, 1127-1137; Eckhoff et al., 2000, Transplantation 69, 1867-1872; Ekberg et al., 2000, Transpl. Int. 13, 151-159). Additional immunosuppressant agents include but are not limited to anti-CD2 (Branco et al., 1999, Transplantation 68, 1588-1596; Przepiorka et al., 1998, Blood 92, 4066-4071), anti-CD4 (Marinova Mutafchieva et al., 2000, Arthritis Rheum. 43, 638-644; Fishwild et al., 1999, Clin. Immunol. 92, 138-152), and anti-CD40 ligand (Hong et al., 2000, Semin. Nephrol. 20, 108-125; Chirmule et al., 2000, J. Virol. 74, 3345-3352; Ito et al., 2000, J. Immunol. 164, 1230-1235).

In some embodiments of the invention, any combination of immunosuppressant agents known to the skilled artisan can be used together with a combination therapy of the invention. One immunosuppressant agent combination of particular utility is tacrolimus (FK506) plus sirolimus (rapamycin) plus daclizumab (anti-IL2 receptor α-subunit antibody). This combination is proven effective as an alternative to steroids and cyclosporine, and when specifically targeting the liver. Moreover, this combination has recently been shown to permit successful pancreatic islet cell transplants. See Denise Grady, The New York Times, Saturday, May 27, 2000, pages A1 and A11. See also A. M. Shapiro et al., Jul. 27, 2000, “Islet Transplantation In Seven Patients With Type 1 Diabetes Mellitus Using A Glucocorticoid Free Immunosuppressive Regimen”, N. Engl. J. Med. 343, 230-238; Ryan et al., 2001, Diabetes 50, 710-719. Plasmaphoresis by any method known in the art may also be used to remove or deplete antibodies that may develop against various components of a combination therapy.

Immune status indicators of use with the invention include but are not limited to antibodies and any of the cytokines known to the skilled artisan, e.g., the interleukins, CSFs and interferons (see generally, Leonard et al., 2000, J. Allergy Clin. Immunol. 105, 877-888; Oberholzer et al., 2000, Crit. Care Med. 28 (4 Suppl.), N3-N12; Rubinstein et al., 1998, Cytokine Growth Factor Rev. 9, 175-181). For example, antibodies specifically immunoreactive with the replacement enzyme or vector components can be monitored to determine immune status of the subject. Among the two dozen or so interleukins known, some immune status indicators include IL-1α, IL-2, IL-4, IL-8 and IL-10. Among the colony stimulating factors (CSFs), immune status indicators include G-CSF, GM-CSF and M-CSF. Among the interferons, one or more alpha, beta or gamma interferons may be used as immune status indicators.

In Sections B through H which follow, various components which may be used for eight specific lysosomal storage diseases are provided (i.e. Gaucher, Fabry, Niemann-Pick B, Hunter, Morquio, Maroteaux-Lamy, Pompe, and Hurler-Scheie). In Section I and subsequent sections, further enabling disclosure for gene therapy, enzyme replacement therapy, and small molecule therapy components of a combination therapy of the invention are provided. A. Gaucher

As noted above, Gaucher's disease is caused by inactivation of the enzyme glucocerebrosidase (β-D-glucosyl-N-acylsphingosine glucohydrolase, EC 3.2.1.45) and accumulation of glucocerebroside (glucosylceramide). For an enzyme replacement therapy component of a combination therapy of the invention for the treatment of Gaucher's disease, a number of references are available which set forth satisfactory dosage regimens and other useful information relating to treatment (see Morales, 1996, Gaucher's Disease: A Review, The Annals of Pharmacotherapy 30, 381-388; Rosenthal et al., 1995, Enzyme Replacement Therapy for Gaucher Disease: Skeletal Responses to Macrophage-targeted Glucocerebrosidase, Pediatrics 96, 629-637; Barton et al., 1991, Replacement Therapy for Inherited Enzyme Deficiency—Macrophage-targeted Glucocerebrosidase for Gaucher's Disease, New England Journal of Medicine 324, 1464-1470; Grabowski et al., 1995, Enzyme Therapy in Type 1 Gaucher Disease: Comparative Efficacy of Mannose-terminated Glucocerebrosidase from Natural and Recombinant Sources, Annals of Internal Medicine 122, 33-39; Pastores et al., 1993, Enzyme Therapy in Gaucher Disease Type 1: Dosage Efficacy and Adverse Effects in 33 Patients treated for 6 to 24 Months, Blood 82, 408-416).

In one embodiment, an ERT dosage regimen of from 2.5 units per kilogram (U/kg) three times a week to 60 U/kg once every two weeks is provided, where the enzyme is administered by intravenous infusion over 1-2 hours. A unit of glucocerebrosidase is defined as the amount of enzyme that catalyzes the hydrolysis of one micromole of the synthetic substrate para-nitrophenyl-β-D-glucopyranoside per minute at 37° C. In another embodiment, a dosage regimen of from 1 U/kg three times a week to 120 U/kg once every two weeks is provided. In yet another embodiment, a dosage regimen of from 0.25 U/kg daily or three times a week to 600 U/kg once every two to six weeks is provided.

Since 1991, aglucerase (CeredaseJ) has been available from Genzyme Corporation. Aglucerase is a placentally-derived modified form of glucocerebrosidase. In 1994, imiglucerase (Cerezyme™) also became available from Genzyme Corporation. Imiglucerase is a modified form of glucocerebrosidase derived from expression of recombinant DNA in a mammalian cell culture system (Chinese hamster ovary cells). Imiglucerase is a monomeric glycoprotein of 497 amino acids containing four N-linked glycosylation sites. Imiglucerase has the advantages of a theoretically unlimited supply and a reduced chance of biological contaminants relative to placentally-derived aglucerase. Both enzymes are modified at their glycosylation sites to expose mannose residues, a maneuver which improves lysosomal targeting via the mannose-6-phosphate receptor. Imiglucerase differs from placental glucocerebrosidase by one amino acid at position 495 where histidine is substituted for arginine. Several dosage regimens of these products are known to be effective (see Morales, 1996, Id.; Rosenthal et al., 1995, Id.; Barton et al., 1991, Id.; Grabowski et al., 1995, Id.; Pastores et al., 1993, Id.). For example, a dosage regimen of 60 U/kg once every two weeks is of clinical benefit in subjects with moderate to severe disease. The references cited above and the package inserts for these products should be consulted by the skilled practitioner for additional dosage regimen and administration information. See also U.S. Pat. Nos. 5,236,838 and 5,549,892 assigned to Genzyme Corporation.

For a small molecule therapy component of a combination therapy of the invention for the treatment of Gaucher's disease, Cox and colleagues provide specific guidance regarding satisfactory dosage regimens and other useful information relating to oral treatment with N-butyldeoxynojirimycin (NB-DNJ) in Gaucher's disease (Cox et al., 2000, Lancet 355, 1481-1485). Additional guidance is provided by the following references relating to various deoxynojirimycin (DNJ)-like compounds: Jeyakumar et al., 2001, Blood 97, 327-329 (NB-DNJ therapy plus bone marrow transplantation); Andersson et al., 2000, Biochem. Pharmacol. 59, 821-829 (N-butyldeoxygalactonojirimycin as a more selective inhibitor than NB-DNJ); Jeyakumar et al., 1999, Proc. Natl. Acad. Sci. USA 96, 6388-6393 (NB-DNJ for treatment of glycosphingolipid storage diseases having a CNS component); and Platt et al., 1997, Science 276, 428-431 (CNS benefit using NB-DNJ to achieve substrate deprivation).

B. Fabry

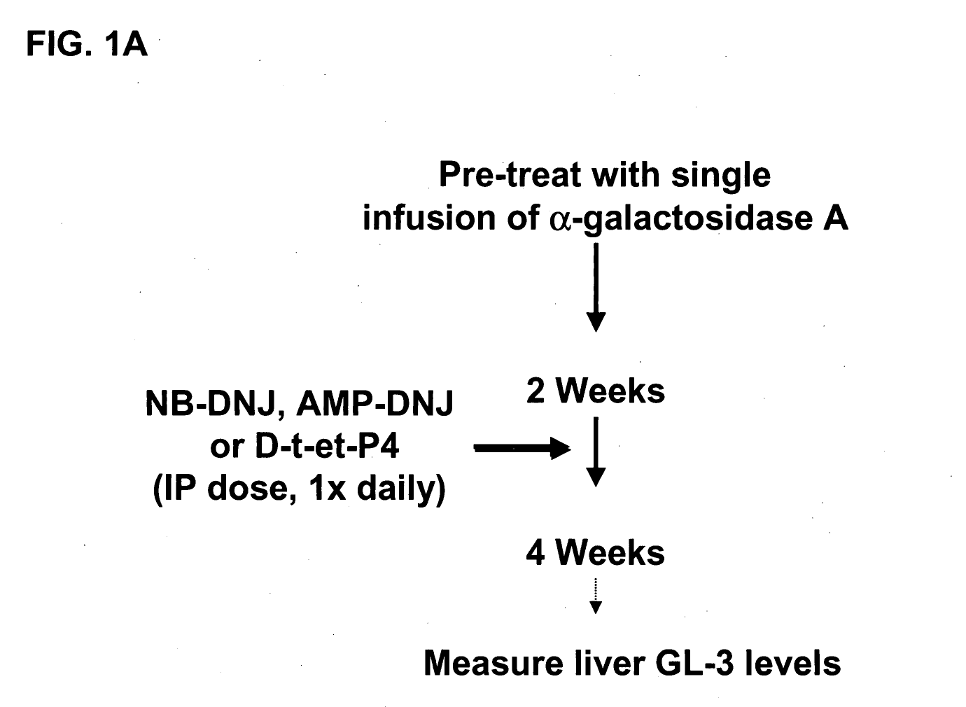

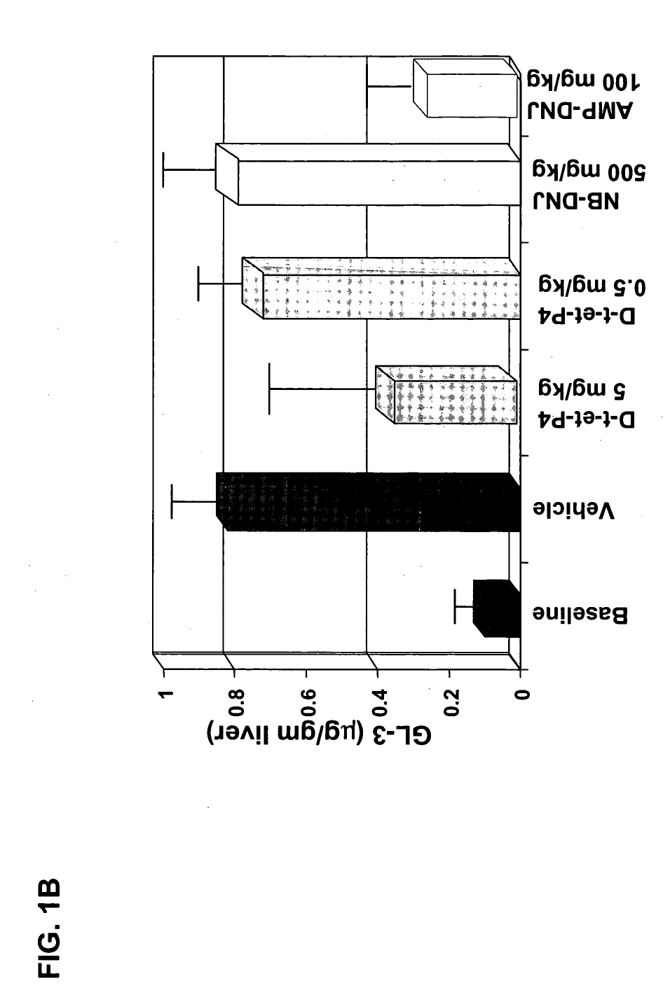

As noted previously, Fabry's disease is caused by inactivation of the lysosomal enzyme alpha-galactosidase A. The enzymatic defect leads to systemic deposition of glycosphingolipids having terminal alpha-galactosyl moieties, predominantly globotriaosylceramide (GL-3 or GL3, see FIG. 1) and, to a lesser extent, galabiosylceramide and blood group B glycosphingolipids.

Several assays are available to monitor disease progression and to determine when to switch from one treatment modality to another. In one embodiment, an assay to determine the specific activity of alpha-galactosidase A in a tissue sample may be used. In another embodiment, an assay to determine the accumulation of GL-3 may be used. In another embodiment, the practitioner may assay for deposition of glycosphingolipid substrates in body fluids and in lysosomes of vascular endothelial, perithelial and smooth muscle cells of blood vessels. Other clinical manifestations which may be useful indicators of disease management include proteinuria, or other signs of renal impairment such as red cells or lipid globules in the urine, and elevated erythrocyte sedimentation rate. One can also monitor anemia, decreased serum iron concentration, high concentration of beta-thromboglobulin, and elevated reticulocyte counts or platelet aggregation. Indeed, any approach for monitoring disease progression which is known to the skilled artisan may be used (See generally Desnick R J et al., 1995, α-Galactosidase A Deficiency: Fabry Disease, In: The Metabolic and Molecular Bases of Inherited Disease, Scriver et al., eds., McGraw-Hill, N.Y., 7th ed., pages 2741-2784).

One surrogate marker is pain for monitoring Fabry disease management. Other methods include the measurement of total clearance of the enzyme and/or substrate from a bodily fluid or biopsy specimen.

One dosage regimen for enzyme replacement therapy in Fabry disease is 1-10 mg/kg i.v. every other day. A dosage regimen from 0.1 to 100 mg/kg i.v. at a frequency of from every other day to once weekly or every two weeks can be used.

In one embodiment, alpha-galactosidase A is provided in Fabry disease using the recombinant viral and/or non viral vectors described in U.S. Pat. No. 6,066,626.

C. Niemann-Pick B

As previously noted, Niemann-Pick B disease is caused by reduced activity of the lysosomal enzyme sphingomyelinase and accumulation of membrane lipid, primarily sphingomyelin. An effective dosage of replacement sphingomyelinase to be delivered may range from about 1 to about 10 mg/kg body weight at a frequency of from every other day to weekly or bi-weekly.

D. Hunter

Hunter's disease (a.k.a. MPS II) is caused by inactivation of iduronate sulfatase and accumulation of dermatan sulfate and heparan sulfate. Hunter=s disease presents clinically in severe and mild forms.

A dosage regimen of therapeutic enzyme from 1 mg/kg every two weeks to 50 mg/kg every week is used in some embodiments.

E. Morquio

Morquio's syndrome (a.k.a. MPS IV) results from accumulation of keratan sulfate due to inactivation of either of two enzymes. In MPS IVA the inactivated enzyme is galactosamine-6-sulfatase and in MPS IVB the inactivated enzyme is beta-galactosidase.

A dosage regimen of therapeutic enzyme from 1 mg/kg every two weeks to 50 mg/kg every week is used in some embodiments.

F. Maroteaux-Lamy

Maroteaux-Lamy syndrome (a.k.a. MPS VI) is caused by inactivation of galactosamine-4-sulfatase (arylsulfatase B) and accumulation of dermatan sulfate.

A dosage regimen of from 1 mg/kg every two weeks to 50 mg/kg every week is one range of effective therapeutic enzyme provided by ERT. Optimally, the dosage employed is less than or equal to 10 mg/kg per week.

One surrogate marker for MPS VI disease progression is proteoglycan levels.

G. Pompe

Pompe's disease is caused by inactivation of the acid alpha-glucosidase enzyme and accumulation of glycogen. The acid alphα-glucosidase gene resides on human chromosome 17 and is designated GM. H. G. Hers first proposed the concept of inborn lysosomal disease based on his studies of this disease, which he referred to as type II glycogen storage disease (GSD II) and which is now also termed acid maltase deficiency (AMD) (see Hers, 1965, Gastroenterology 48, 625).

Several assays are available to monitor Pompe disease progression. Any assay known to the skilled artisan may be used. For example, one can assay for intra-lysosomal accumulation of glycogen granules, particularly in myocardium, liver and skeletal muscle fibers obtained from biopsy. Alphα-glucosidase enzyme activity can also be monitored in biopsy specimens or cultured cells obtained from peripheral blood. Serum elevation of creatine kinase (CK) can be monitored as an indication of disease progression. Serum CK can be elevated up to ten-fold in infantile-onset patients and is usually elevated to a lesser degree in adult-onset patients. See Hirschhorn R, 1995, Glycogen Storage Disease Type II: Acid α-Glucosidase (Acid Maltase) Deficiency, In: The Metabolic and Molecular Bases of Inherited Disease, Scriver et al., eds., McGraw-Hill, N.Y., 7th ed., pages 2443-2464.

H. Hurler-Scheie

Hurler, Scheie, and Hurler-Scheie disease, also known as MPS I, are caused by inactivation of alpha-iduronidase and accumulation of dermatan sulfate and heparan sulfate.

Several assays are available to monitor MPS I disease progression. For example, alpha-iduronidase enzyme activity can be monitored in tissue biopsy specimens or cultured cells obtained from peripheral blood. In addition, a convenient measure of disease progression in MPS I and other mucopolysaccharidoses is the urinary excretion of the glycosaminoglycans dermatan sulfate and heparan sulfate (see Neufeld et al., 1995, Id.).

I. Gene Therapy

One of the most frequently used methods for administration of gene therapy, both in vivo and ex vivo, is the use of viral vectors for delivery of the gene. Many species of virus are known, and many have been extensively studied for gene therapy purposes. The most commonly used viral vectors include those derived from adenovirus, adeno-associated virus (AAV) and retrovirus, including lentivirus such as human immunodeficiency virus (HIV). See also WO 99/57296 and WO 99/41399.

Among adenovirus, pseudoadenovirus (PAV or gutless adenovirus) is used in one embodiment (see below). In this group of vectors, a titre range of from 10⁹ to 10¹³ particles per kg body weight may be used for administration to a subject. For AAV, a titre range of from 10⁹ to 10¹⁴ particles per kg body weight may be used for administration to a subject. For lentivirus, a titre range of from 10⁶ to 10¹⁰ particles per kg body weight may be used for administration to a subject. In each instance, the exact titre is determined by adjusting the titre to the amount necessary to deliver an effective amount of enzyme.

1. Adenovirus

Adenoviral vectors for use to deliver transgenes to cells for various applications, such as in vivo gene therapy and in vitro study and/or production of the products of transgenes, are commonly derived from adenoviruses by deletion of the early region 1 (E1) genes (Berkner, K. L., 1992, Curr. Top. Micro. Immunol. 158, 39-66). Deletion of E1 genes renders such adenoviral vectors replication defective and significantly reduces expression of the remaining viral genes present within the vector. However, it is believed that the presence of the remaining viral genes in adenoviral vectors can be deleterious to the transfected cell for one or more of the following reasons: (1) stimulation of a cellular immune response directed against expressed viral proteins; (2) cytotoxicity of expressed viral proteins; and (3) replication of the vector genome leading to cell death.

One solution to this problem has been the creation of adenoviral vectors with deletions of various adenoviral gene sequences. In particular, pseudoadenoviral vectors (PAVs), also known as ‘gutless adenovirus’ or mini-adenoviral vectors, are adenoviral vectors derived from the genome of an adenovirus that contain minimal cis-acting nucleotide sequences required for the replication and packaging of the vector genome and which can contain one or more transgenes (see U.S. Pat. No. 5,882,877 by Gregory et al. which covers pseudoadenoviral vectors (PAV) and methods for producing PAV). Such PAVs, which can accommodate up to about 36 kb of foreign nucleic acid, are advantageous because the carrying capacity of the vector is optimized while the potential for host immune responses to the vector or the generation of replication-competent viruses is reduced. PAV vectors contain the 5′ inverted terminal repeat (ITR) and the 3′ ITR nucleotide sequences that contain the origin of replication, and the cis-acting nucleotide sequence required for packaging of the PAV genome, and can accommodate one or more transgenes with appropriate regulatory elements, e.g., promoters, enhancers, etc.

Adenoviral vectors, such as PAVs, have been designed to take advantage of the desirable features of adenovirus which render it a suitable vehicle for delivery of nucleic acids to recipient cells. Adenovirus is a non-enveloped, nuclear DNA virus with a genome of about 36 kb, which has been well-characterized through studies in classical genetics and molecular biology (Hurwitz, M. S., Adenoviruses, Virology, 3rd edition, Fields et al.; eds., Raven Press, New York, 1996; Hitt, M. M. et al., Adenovirus Vectors, The Development of Human Gene Therapy, Friedman, T. ed., Cold Spring Harbor Laboratory Press, New York, 1999). The viral genes are classified into early (designated E1-E4) and late (designated L1-L5) transcriptional units, referring to the generation of two temporal classes of viral proteins. The demarcation of these events is viral DNA replication. The human adenoviruses are divided into numerous serotypes (approximately 47, numbered accordingly and classified into 6 groups: A, B, C, D, E and F), based upon properties including hemagglutination of red blood cells, oncogenicity, DNA and protein amino acid compositions and homologies, and antigenic relationships.

Recombinant adenoviral vectors have several advantages for use as gene delivery vehicles, including tropism for both dividing and non-dividing cells, minimal pathogenic potential, ability to replicate to high titer for preparation of vector stocks, and the potential to carry large inserts (Berkner, K. L., Curr. Top. Micro. Immunol. 158:39-66, 1992; Jolly, D., Cancer Gene Therapy 1:51-64,1994).

PAVs have been designed to take advantage of the desirable features of adenovirus which render it a suitable vehicle for gene delivery. While adenoviral vectors can generally carry inserts of up to 8 kb in size by the deletion of regions which are dispensable for viral growth, maximal carrying capacity can be achieved with the use of adenoviral vectors containing deletions of most viral coding sequences, including PAVs. See U.S. Pat. No. 5,882,877 by Gregory et al.; Kochanek et al., Proc. Natl. Acad. Sci. USA 93:5731-5736,1996; Parks et al., Proc. Natl. Acad. Sci. USA 93:13565-13570,1996; Lieber et al., J. Virol. 70:8944-8960, 1996; Fisher et al., Virology 217:11-22, 1996; U.S. Pat. No. 5,670,488; PCT Publication No. WO 96/33280, published Oct. 24, 1996; PCT Publication No. WO 96/40955, published Dec. 19, 1996; PCT Publication No. WO 97/25446, published Jul. 19, 1997; PCT Publication No. WO 95/29993, published Nov. 9, 1995; PCT Publication No. WO 97/00326, published Jan. 3, 1997; Morral et al., Hum. Gene Ther. 10:2709-2716, 1998.

Since PAVs are deleted for most of the adenovirus genome, production of PAVs requires the furnishing of adenovirus proteins in trans which facilitate the replication and packaging of a PAV genome into viral vector particles. Most commonly, such proteins are provided by infecting a producer cell with a helper adenovirus containing the genes encoding such proteins. However, such helper viruses are potential sources of contamination of a PAV stock during purification and can pose potential problems when administering the PAV to an individual if the contaminating helper adenovirus can replicate and be packaged into viral particles.

Accordingly, it is advantageous to increase the purity of a PAV stock by reducing or eliminating any production of helper vectors which can contaminate preparation. Several strategies to reduce the production of helper vectors in the preparation of a PAV stock are disclosed in U.S. Pat. No. 5,882,877, issued Mar. 16, 1999; U.S. Pat. No. 5,670,488, issued Sep. 23, 1997 and International Patent Application No. PCT/US99/03483. For example, the helper vector may contain: (a) mutations in the packaging sequence of its genome to prevent its packaging; (b) an oversized adenoviral genome which cannot be packaged due to size constraints of the virion; or (c) a packaging signal region with binding sequences that prevent access by packaging proteins to this signal which thereby prevents production of the helper virus. Other strategies include the design of a helper virus with a packaging signal flanked by the excision target site of a recombinase, such as the Cre-Lox system (Parks et al., Proc. Natl. Acad. Sci. USA 93: 13565-13570, 1996; Hardy et al., J. Virol. 71:1842-1849, 1997). Such helper vectors reduce the yield of wild-type levels.

The use of adenoviruses for gene therapy is described, for example, in U.S. Pat. Nos. 6,040,174; 5,882,877; 5,824,544; 5,707,618; and 5,670,488.

2. Adeno-Associated Virus (AAV)

Adeno-associated virus (AAV) is a single-stranded human DNA parvovirus whose genome has a size about of 4.6 kb. The AAV genome contains two major genes: the rep gene, which codes for the rep proteins (Rep 76, Rep 68, Rep 52 and Rep 40) and the cap gene, which codes for AAV structural proteins (VP-1, VP-2 and VP-3). The rep proteins are involved in AAV replication, rescue, transcription and integration, while the cap proteins form the AAV viral particle. AAV derives its name from its dependence on an adenovirus or other helper virus (e.g. herpesvirus) to supply essential gene products that allow AAV to undergo a productive infection, i.e., reproduce itself in the host cell. In the absence of helper virus, AAV integrates as a provirus into the host cell's chromosome, until it is rescued by superinfection of the host cell with a helper virus, usually adenovirus (Muzyczka, 1992, Curr. Top. Micro. Immunol. 158:97).

Utility of AAV as a gene transfer vector results from several unique features of its biology. At both ends of the AAV genome is a nucleotide sequence, known as an inverted terminal repeat (ITR), which contains the cis-acting nucleotide sequences required for virus replication, rescue, packaging and integration. The integration function of the ITR mediated by the rep protein in trans permits the AAV genome to integrate into a cellular chromosome after infection, in the absence of helper virus. This unique property of the virus has relevance to the use of AAV in gene transfer, as it allows for integration of a recombinant AAV (rAAV) containing a gene of interest into the cellular genome. Therefore, stable genetic transformation, ideal for many of the goals of gene transfer, may be achieved by use of rAAV vectors. Furthermore, the site of integration for AAV is well-established and has been localized to chromosome 19 of humans (Kotin et al., Proc. Natl. Acad. Sci. 87:2211-2215,1990). This predictability of integration site reduces the danger of random insertional events into the cellular genome that may activate or inactivate host genes or interrupt coding sequences, consequences that can limit the use of vectors whose integration is random, e.g., retroviruses. However, because the rep protein mediates the integration of AAV, removal of this gene in the design of rAAV vectors may result in the altered integration patterns that have been observed with rAAV vectors (Ponnazhagan et al., Hum. Gene Ther. 8:275-284, 1997).

There are other advantages to the use of AAV for gene transfer. The host range of AAV is broad. Moreover, unlike retroviruses, AAV can infect both quiescent and dividing cells. In addition, AAV has not been associated with human disease, obviating many of the concerns that have been raised with retrovirus-derived gene transfer vectors.

Any known AAV serotype may be used as a gene therapy vector, e.g., AAV1, AAV2, AAV5, AAV7 and AAV8.

Standard approaches to the generation of recombinant AAV vectors have required the coordination of a series of intracellular events: transfection of the host cell with an rAAV vector genome containing a transgene of interest flanked by the AAV ITR sequences, transfection of the host cell by a plasmid encoding the genes for the AAV rep and cap proteins which are required in trans, and infection of the transfected cell with a helper virus to supply the non-AAV helper functions required in trans (Muzyczka, N., Curr. Top. Micro. Immuhol. 158: 97-129,1992). The adenoviral (or other helper virus) proteins activate transcription of the AAV rep gene, and the rep proteins then activate transcription of the AAV cap genes. The cap proteins then utilize the ITR sequences to package the rAAV genome into an rAAV viral particle. Therefore, the efficiency of packaging is determined, in part, by the availability of adequate amounts of the structural proteins, as well as by the accessibility of any cis-acting packaging sequences required in the rAAV vector genome.

One of the potential limitations to high level rAAV production derives from limiting quantities of the AAV helper proteins required in trans for replication and packaging of the rAAV genome. Some approaches to increasing the levels of these proteins have included the following: placing the AAV rep gene under the control of the HIV LTR promoter to increase rep protein levels (Flotte, F. R. et al., Gene Therapy 2:29-37, 1995); the use of other heterologous promoters to increase expression of the AAV helper proteins, specifically the cap proteins (Vincent et al., J. Virol. 71:1897-1905, 1997); and the development of cell lines that specifically express the rep proteins (Yang, Q. et al., J. Virol. 68: 4847-4856, 1994).

Other approaches to improving the production of rAAV vectors include the use of helper virus induction of the AAV helper proteins (Clark et al., Gene Therapy 3:1124-1132, 1996) and the generation of a cell line containing integrated copies of the rAAV vector and AAV helper genes so that infection by the helper virus initiates rAAV production (Clark et al., Human Gene Therapy 6:1329-1341, 1995).

rAAV vectors have been produced using replication-defective helper adenoviruses which contain the nucleotide sequences encoding the rAAV vector genome (U.S. Pat. No. 5,856,152 issued Jan. 5, 1999) or helper adenoviruses which contain the nucleotide sequences encoding the AAV helper proteins (PCT International Publication WO 95/06743, published Mar. 9, 1995). Production strategies which combine high level expression of the AAV helper genes and the optimal choice of cis-acting nucleotide sequences in the rAAV vector genome have been described (PCT International Application No. WO97/09441 published Mar. 13, 1997).

Current approaches to reducing contamination of rAAV vector stocks by helper viruses, therefore, involve the use of temperature-sensitive helper viruses (Ensinger et al., J.Virol. 10:328-339, 1972), which are inactivated at the non-permissive temperature. Alternatively, the non-AAV helper genes can be subcloned into DNA plasmids which are transfected into a cell during rAAV vector production (Salvetti et al., Hum. Gene Ther. 9:695-706,1998; Grimm et al., Hum. Gene Ther. 9:2745-2760, 1998).

The use of AAV for gene therapy is described, for example, in U.S. Pat. Nos. 5,753,500 and 5,962,313.

3. Retrovirus

Retrovirus vectors are a common tool for gene delivery (Miller, 1992, Nature 357, 455-460). The ability of retrovirus vectors to deliver an un-rearranged, single copy gene into a broad range of rodent, primate and human somatic cells makes retroviral vectors well suited for transferring genes to a cell.

Retroviruses are RNA viruses wherein the viral genome is RNA. When a host cell is infected with a retrovirus, the genomic RNA is reverse transcribed into a DNA intermediate which is integrated very efficiently into the chromosomal DNA of infected cells. This integrated DNA intermediate is referred to as a provirus. Transcription of the provirus and assembly into infectious virus occurs in the presence of an appropriate helper virus or in a cell line containing appropriate sequences enabling encapsidation without coincident production of a contaminating helper virus. A helper virus is not required for the production of the recombinant retrovirus if the sequences for encapsidation are provided by co-transfection with appropriate vectors.

Another useful tool for producing recombinant retroviral vectors is a packaging cell line which supplies in trans the proteins necessary for producing infectious virions but which is incapable of packaging endogenous viral genomic nucleic acids (Watanabe and Temin, 1983, Molec. Cell. Biol. 3(12):2241-2249; Mann et al., 1983, Cell 33:153-159; Embretson and Temin, 1987, J. Virol. 61(9):2675-2683). One approach to minimize the likelihood of generating replication competent retrovirus (RCR) in packaging cells is to divide the packaging functions into two genomes. For example, one genome may be used to express the gag and pol gene products and the other to express the env gene product (Bosselman et al., 1987, Molec. Cell. Biol. 7(5):1797-1806; Markowitz et al., 1988, J. Virol. 62(4):1120-1124; Danos and Mulligan, 1988, Proc. Natl. Acad. Sci. 85:6460-6464). This approach minimizes the possibility that co-packaging and subsequent transfer of the two genomes will occur; it also significantly decreases the frequency of recombination to produce RCR due to the presence of three retroviral genomes in the packaging cell.

In the event recombinants arise, mutations (Danos and Mulligan, 1988, Id.) or deletions (Bosselman et al., 1987, Id.; Markowitz et al., 1988, Id.) can be configured within the undesired gene products to render any possible recombinants non-functional. In addition, deletion of the 3′ LTR on both packaging constructs further reduces the ability to form functional recombinants.

The retroviral genome and the proviral DNA have three genes: the gag, the pol, and the env, which are flanked by two long terminal repeat (LTR) sequences. The gag gene encodes the internal structural (matrix, capsid, and nucleocapsid) proteins; the pol gene encodes the RNA-directed DNA polymerase (reverse transcriptase) and the env gene encodes viral envelope glycoproteins. The 5′ and 3′ LTRs serve to promote transcription and polyadenylation of the virion RNAs. The LTR contains all other cis-acting sequences necessary for viral replication. Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the result is a cis defect which prevents encapsidation of genomic RNA. However, the resulting mutant is still capable of directing the synthesis of all virion proteins.

Lentiviruses are complex retroviruses which, in addition to the common retroviral genes gag, pol and env, contain other genes with regulatory or structural function. For example, lentiviruses may have additional genes including vit, vpr, tat, rev, vpu, nef, and vpx. The higher complexity enables the lentivirus to modulate the life cycle, as in the course of latent infection. A typical lentivirus is the human immunodeficiency virus (HIV), the etiologic agent of AIDS. In vivo, HIV can infect terminally differentiated cells that rarely divide, such as lymphocytes and macrophages. In vitro, HIV can infect primary cultures of monocyte-derived macrophages (MDM) as well as HeLa-Cd4 or T lymphoid cells arrested in the cell cycle by treatment with aphidicolin or gamma irradiation. Infection of cells is dependent on the active nuclear import of HIV preintegration complexes through the nuclear pores of the target cells. That occurs by the interaction of multiple, partly redundant, molecular determinants in the complex with the nuclear import machinery of the target cell. Identified determinants include a functional nuclear localization signal (NLS) in the gag matrix (MA) protein, the karyophilic virion-associated protein, vpr, and a C-terminal phosphotyrosine residue in the gag MA protein.

The use of retroviruses for gene therapy is described, for example, in U.S. Pat. Nos. 6,013,516 and 5,994,136.

4. Non-Viral Vector

Additional methods for delivery of DNA to cells do not use viruses for delivery. Such methods include the use of compounds such as cationic amphiphilic compounds, non-viral ex vivo transfection, as well as DNA in the absence of viral or non-viral compounds, known as “naked DNA.”

Because compounds designed to facilitate intracellular delivery of biologically active molecules must interact with both non-polar and polar environments (in or on, for example, the plasma membrane, tissue fluids, compartments within the cell, and the biologically active molecule itself), such compounds are designed typically to contain both polar and non-polar domains. Compounds having both such domains may be termed amphiphiles, and many lipids and synthetic lipids that have been disclosed for use in facilitating such intracellular delivery (whether for in vitro or in vivo application) meet this definition. One particularly important class of such amphiphiles is the cationic amphiphiles. In general, cationic amphiphiles have polar groups that are capable of being positively charged at or around physiologic pH, and this property is understood in the art to be important in defining how the amphiphiles interact with the many types of biologically active (therapeutic) molecules including, for example, negatively charged polynucleotides such as DNA.

Examples of cationic amphiphilic compounds that have both polar and non-polar domains and that are stated to be useful in relation to intracellular delivery of biologically active molecules are found, for example, in the following references, which references also contain useful discussion of (1) the properties of such compounds that are understood in the art as making them suitable for such applications, and (2) the nature of the structures, as understood in the art, that are formed by complexing of such amphiphiles with therapeutic molecules intended for intracellular delivery. Feigner, et al., Proc. Natl. Acad. Sci. USA, 84, 7413-7417 (1987) disclose use of positively-charged synthetic cationic lipids including N-[1(2,3-dioleyloxy)propyl]-N,N, N-trimethylammonium chloride (“DOTMA”), to form lipid/DNA complexes suitable for transfections. See also Feigner et al., 1994, J. Biol. Chem. 269, 2550-2561. Behr et al., Proc. Natl. Acad. Sci. USA, 86, 6982-6986 (1989) disclose numerous amphiphiles including dioctadecylamidologlycylspermine (“DOGS”). U.S. Pat. No. 5,283,185 to Epand et al. describes additional classes and species of amphiphiles including 3β[N-(N¹,N¹-dimethylaminoethane)-carbamoyl] cholesterol, termed “DC-chol”. Additional compounds that facilitate transport of biologically active molecules into cells are disclosed in U.S. Pat. No. 5,264,618 to Felgner et al. See also Felgner et al., 1994, J. Biol. Chem. 269, 2550-2561, for disclosure therein of further compounds including “DMRIE” or 1,2-dimyristyloxypropyl-3-dimethyl-hydroxyethyl ammonium bromide. Reference to amphiphiles suitable for intracellular delivery of biologically active molecules is also found in U.S. Pat. No. 5,334,761 to Gebeyehu et al., and in Felgner et al., 1993, Meth. Enzymol. 5, 67-75.

The use of compositions comprising cationic amphiphilic compounds for gene delivery is described, for example, in U.S. Pat. Nos. 5,049,386; 5,279,833; 5,650,096; 5,747,471; 5,757,471; 5,767,099; 5,910,487; 5,719,131; 5,840,710; 5,783,565; 5,925,628; 5,912,239; 5,942,634; 5,948,925; 6,022,874; 5,994,317; 5,861,397; 5,952,916; 5,948,767; 5,939,401; and 5,935,936.

Another approach to gene therapy is the non-viral transfection ex vivo of a primary or secondary host cell derived from a subject to be treated with a DNA construct carrying the therapeutic gene. Host cells engineered in this way are then re-introduced into the subject to administer the gene therapy. See, e.g., U.S. Pat. Nos. 5,994,127; 6,048,524; 6,048,724; 6,048,729; 6,054,288; and 6,063,630.

Methods for delivering a non-infectious, non-integrating DNA sequence encoding a desired polypeptide or peptide operably linked to a promoter, free from association with transfection-facilitating proteins, viral particles, liposomal formulations, charged lipids and calcium phosphate precipitating agents, is described in U.S. Pat. Nos. 5,580,859; 5,963,622; and 5,910,488.

Gene transfer systems that combine viral and nonviral components have been developed. See Cristiano et al., 1993, Proc. Natl. Acad. Sci. USA 90, 11548; Wu et al., 1994, J. Biol. Chem. 269, 11542; Wagner et al., 1992, Proc. Natl. Acad. Sci. USA 89, 6099; Yoshimura et al., 1993, J. Biol. Chem. 268, 2300; Curiel et al., 1991, Proc. Natl. Acad. Sci USA 88, 8850; Kupfer et al., 1994, Hum. Gene Ther. 5,1437; and Gottschalk et al., 1994, Gene Ther. 1,185. In most cases, adenovirus has been incorporated into the gene delivery systems to take advantage of its endosomolytic properties. The reported combinations of viral and nonviral components generally involve either covalent attachment of the adenovirus to a gene delivery complex or co-internalization of unbound adenovirus with cationic lipid: DNA complexes.

5. Regulated Gene Expression

A number of systems are available to provide regulated expression of a gene delivered to a subject. Any such system known to the skilled artisan may be used in a combination therapy of the invention. Examples of such systems include but are not limited to tet-regulated vectors (see e.g. U.S. Pat. Nos. 6,004,941 and 5,866,755), RU486 gene regulation technology (see U.S. Pat. Nos. 5,874,534 and 5,935,934), and modified FK506 gene regulation technology (see U.S. Pat. Nos. 6,011,018; 5,994,313; 5,871,753; 5,869,337; 5,834,266; 5,830,462; WO 96/41865; and WO 95/33052).

J. Enzyme Replacement Therapy

The following sections set forth specific disclosure and alternative embodiments available for the enzyme replacement therapy component of a combination therapy of the invention.

Generally, dosage regimens for an enzyme replacement therapy component of a combination therapy of the invention are generally determined by the skilled clinician. Several examples of dosage regimens for the treatment of Gaucher's disease with glucocerebrosidase were provided above in Section A. The general principles for determining a dosage regimen for any given ERT component of a combination therapy of the invention for the treatment of any LSD will be apparent to the skilled artisan from a review of the specific references cited in the sections which set forth the enabling information for each specific LSD.

Any method known in the art may be used for the manufacture of the enzymes to be used in an enzyme replacement therapy component of a combination therapy of the invention. Many such methods are known and include but are not limited to the Gene Activation technology developed by Transkaryotic Therapies, Inc. (see U.S. Pat. Nos. 5,968,502 and 5,272,071).

K. Small Molecule Therapy

The following section sets forth specific disclosures and alternative embodiments available for the small molecule therapy component of a combination therapy of the invention. Dosage regimens for a small molecule therapy component of a combination therapy of the invention are generally determined by the skilled clinician and are expected to vary significantly depending on the particular storage disease being treated and the clinical status of the particular affected individual. The general principles for determining a dosage regimen for a given SMT component of any combination therapy of the invention for the treatment of any storage disease are well known to the skilled artisan. Guidance for dosage regimens can be obtained from any of the many well known references in the art on this topic. Further guidance is available, inter alia, from a review of the specific references cited herein.

Generally, substrate deprivation inhibitors such as DNJ-type inhibitors and amino ceramide-like compounds (including P4-type inhibitors) may be used in the combination therapies of the invention for treatment of virtually any storage disease resulting from a lesion in the glycosphingolipid pathway (e.g. Gaucher, Fabry, Sandhoff, Tay-Sachs, G_{M1}-gangliosidosis). Likewise, aminoglycosides (e.g. gentamicin, G418) may be used in the combination therapies of the invention for any storage disease individual having a premature stop-codon mutation. Such mutations are particularly prevalent in Hurler syndrome. A small molecule therapy component of a combination therapy of the invention may be used where there is a central nervous system manifestation to the storage disease being treated (e.g. Sandhoff, Tay-Sachs, Niemann-Pick Type>A), since small molecules can generally cross the blood-brain barrier with ease when compared to other therapies. Moreover, derivatives of the small molecules set forth herein are provided, wherein the derivatives have been designed by any method known in the art to facilitate or enhance crossing the blood-brain barrier.

Accordingly, this invention provides small molecule therapy in combination with enzyme replacement therapy and/or gene therapy for treatment of storage diseases. Small molecules useful in the combination therapies of the invention may include but are not limited to those described by Shayman and coworkers, by Aerts and coworkers, and by Bedwell and coworkers in the references cited below.

Examples of amino ceramide-like compounds useful in the combination therapies of the invention may include but are not limited to those described in the following references: Abe et al., 2000, J. Clin. Invest. 105, 1563-1571; Abe et al., 2000, Kidney Int'l 57, 446-454; Lee et al., 1999, J. Biol. Chem. 274, 14662-14669; Shayman et al., 2000, Meth. Enzymol. 31, 373-387; U.S. Pat. Nos. 5,916,911; 5,945,442; 5,952,370; 6,030,995; 6,040,332 and 6,051,598. Compounds include but are not limited to PDMP and its derivatives, wherein PDMP is 1-phenyl-2-decanoylamino-3-morpholino-1-propanol (see U.S. Pat. No. 5,916,911) and P4 and its derivatives, wherein P4 is D-threo-1-phenyl-2-palmitoylamino-3-pyrrolidino-1-propanol (see Lee et al., 1999, id.). P4 derivatives include D-threo-4′-hydroxy-1-phenyl-2-palmitoylamino-3-pyrrolidino-1-propanol (4′-hydroxy-P4), D-threo-1-(3′,4′-trimethylenedioxy)phenyl-2-palmitoylamino-3-pyrrolid ino-1-propanol (trimethylenedioxy-P4), D-threo-1-(3′,4′-methylenedioxy)phenyl-2-palmitoylamino-3-pyrrolidino-1-propanol (methylenedioxy-P4) and D-threo-1-(3′,4′-ethylenedioxy)phenyl-2-palmitoylamino-3-pyrrolidino-1-propanol (ethylenedioxy-P4 or D-t-et-P4). One particular P4 derivative is ethylenedioxy-P4 (see e.g. D-t-et-P4 in FIG. 1).

Dosages of P4 derivatives including D-t-et-P4 in a combination therapy of the invention are easily determined by the skilled artisan. In some embodiments, such dosages may range from 0.5 mg/kg to 50 mg/kg, in other embodiments from 1 mg/kg to 10 mg/kg by intraperitoneal or equivalent administration from one to five times daily. In some embodiments, such dosages may range from 5 mg/kg to 5 g/kg, in other embodiments from 10 mg/kg to 1 g/kg by oral or equivalent administration from one to five times daily in some embodiments an oral dose range for a P4-like compound is from 6 mg/kg/day to 600 mg/kg/day.

Deoxynojirimycin-like compounds and related small molecules are useful in the combination therapies of the invention. N-butyldeoxynojirimycin (NB-DNJ or OGT 918) and derivatives thereof may be used in combination therapies of the invention for treatment of storage diseases in the glycosphingolipid pathway. The use of OGT 918 alone as an oral treatment for Gaucher's disease has been reported by Cox et al., 2000, Lancet 355, 1481-1485. OGT 918 can be used in combination therapies of the invention for any storage disease of the glycosphingolipid pathway, including Sandhoff and Tay-Sachs disease (see e.g. Jeyakumar et al., 2001, Blood 97, 327-329; Andersson et al., 2000, Biochem. Pharmacol. 59, 821-829; Jeyakumar et al., 1999, Proc. Natl. Acad. Sci. USA 96, 6388-6393; and Platt et al., 1997, Science 276, 428-431). Deoxynojirimycin derivatives include but are not limited to N-propyldeoxynojirimycin, N-butyldeoxynojirimycin, N-butyideoxygalactonojirimycin, N-pentlydeoxynojirimycin, N-heptyldeoxynojirimycin, N-pentanoyldeoxynojirimycin, N-(5-adamantane-1-ylmethoxy)pentyl)-deoxynojirimycin, N-(5-cholesteroxypentyl)-deoxynojirimycin, N-(4-adamantanemethanylcarboxy-1-oxo)-deoxynojirimycin, N-(4-adamantanylcarboxy-1-oxo)-deoxynojirimycin, N-(4-phenantrylcarboxy-1-oxo)-deoxynojirimycin, N-(4-cholesterylcarboxy-1-oxo)-deoxynojirimycin, or N-(4-b-cholestanylcarboxy-1-oxo)-deoxynojirimycin.

Deoxynojirimycin derivative for use in the combination therapies of the invention is N-(5-adamantane-1-yl-methoxy)pentyl)-deoxynojirimycin (AMP-DNJ or AMP-DNM, see FIG. 1). AMP-DNJ is among a variety of DNJ derivatives originally designed as research tools to aid in the elucidation of the physiological relevance of the non-lysosomal glucosylceramidase (Overkleeft et al., 1998, J. Biol. Chem. 273, 26522-26527). Another deoxynojirimycin derivative for use in the combination therapies of the invention is N-butyldeoxygalactonojirimycin (NB-DGJ), a DNJ-type inhibitor with greater selectivity (see Andersson et al., 2000, Biochem. Pharmacol. 59, 821-829).

Dosages of DNJ derivatives including NB-DNJ, NB-DGJ, AMP-DNJ in a combination therapy of the invention are also readily determined by the skilled artisan. In some embodiments, such dosages may range from 0.01 mg/kg to 1000 mg/kg, in other embodiments from 0.1 mg/kg to 100 mg/kg, in yet other embodiments, from 1 mg/kg to 10 mg/kg, by intraperitoneal or equivalent administration from one to five times daily. Such dosages, when administered orally, may range from two- to twenty-fold greater. For example, OGT 918 (a.k.a. NB-DNJ) has been administered orally to humans in a 100 mg dose three times per day for twelve months, and a daily dose of up to 3 gm has been used. An oral dose range for a DNJ-like compound is from 60 mg/kg/day to 900 mg/kg/day.

The aminoglycosides such as gentamicin and G418 are particularly useful in the combination therapies of the invention where the affected individual has a storage disease with at least one allele comprising a premature stop-codon mutation. This approach is particularly useful in some Hurler syndrome patient populations, where premature stop mutations represent roughly two-thirds of the disease-causing mutations. The work by Bedwell and coworkers provides guidance for the skilled artisan in the use of stop-mutation suppressors such as the aminoglycosides (U.S. Pat. No. 5,840,702). Aminoglycoside-induced read-through of Hurler syndrome mutations have been described by Keeling et al., 2001, Hum. Molec. Genet. 10, 291-299. Some aminoglycosides which are for use in the combination therapies of the invention include but are not limited to gentamicin, G418, hygromycin B, paromomycin, tobramycin and Lividomycin A.

Dosages of aminoglycoside derivatives including gentamicin and G418 in a combination therapy of the invention are also readily determined by the skilled artisan. In some embodiments dosages may range from 1 mg/kg to 1000 mg/kg, in other embodiments from 10 mg/kg to 100 mg/kg, in yet ot5her embodiments, from 5 mg/kg to 50 mg/kg, by intraperitoneal or equivalent administration from one to five times daily. Such dosages, when administered orally, may range from two- to twenty-fold greater.

Any storage disease resulting at least in part from a premature stop codon can be treated with an aminoglycoside in combination with GT and/or ERT. A number of examples of storage diseases for which premature stop codons have been identified are provided in the following references: Peltola et al., 1994, Hum. Molec. Genet. 3, 2237-2242 (Aspartylglucosaminuria); Voskoboeva et al., 1994, Hum. Genet. 93, 259-64 (Maroteaux-Lamy); Yang et al., 1993, Biochim. Biophys. Acta 1182, 245-9 (Fucosidosis); Takahashi et al., 1992, J. Biol. Chem. 267, 12552-8 (Niemann-Pick); Beutler et al., 1996, Proc. Assoc. Am. Phys. 108, 179-84 (Gaucher); Hara et al., 1994, Hum. Genet. 94, 136-40 (Sandhoff); Zhang et al., 1994, Hum. Molec. Genet. 3, 139-145 (Sandhoff); Tanaka et al., 1999, J. Hum. Genet. 44, 91-5 (Tay-Sachs); Okumiya et al., 1996, Jpn. J. Hum. Genet. 41, 313-21 (Fabry); Drucker et al., 1993, Hum. Mutat. 2, 415-7 (Tay-Sachs); and Sakuraba et al., 1990, Am. J. Hum. Genet. 47, 784-9 (Fabry). To determine whether a storage disease individual will benefit from a combination therapy which includes an aminoglycoside (or any other agent able to elicit read-through), the clinician simply has the individual genotyped to determine whether a premature stop codon mutation is present in one or more disease alleles.

L. Dosing Regimens & Other Considerations

Initially, gene therapy may be used to debulk accumulated lysosomal storage material in affected cells and organs. However, expression from currently-available gene therapy vectors generally extinguishes over time. Accordingly, gene therapy may be followed with recombinant enzyme administration when gene expression begins to decline. ERT may be continued, for example, until the antibody titer against the viral vector being used has abated sufficiently to allow re-dosing with gene therapy. Switching to a different gene therapy vector is also possible. Finally, both the GT and ERT phases of treatment may be supplemented with SMT, as needed, depending on the clinical course of a given storage disease in a given individual.

Alternatively, as expression from a gene therapy vector extinguishes over time, GT may be followed with substrate inhibition therapy (using one or more small molecules) to abate the rate of re-accumulation of storage material. Depending on the rate of re-accumulation, patients can be re-treated with gene therapy (when immune status indicators indicate it is safe to do so) or with enzyme therapy. The intervening period between gene therapy and substrate inhibition and/or enzyme therapy is dictated by storage disease type and severity. Individuals which have lysosomal storage disorders that accumulate storage material slowly over time, or those which have relatively high levels of residual enzyme activity, will require less-frequent re-treatment with gene therapy at longer intervals.

Enzyme therapy can also be used initially to debulk accumulated lysosomal storage in affected cells and organs. After debulking, subjects may receive substrate inhibition therapy to abate the rate of re-accumulation of storage material in affected lysosomes. The re-accumulation rate will vary, depending on disease type and severity, and subjects can subsequently receive re-treatment with enzyme therapy, or with gene therapy, as needed as determined by the skilled clinician.

After enzyme therapy debulking, subjects may alternatively be treated with gene therapy which could provide therapeutic levels of enzyme for several months. As expression expires, subjects may return to enzyme therapy or receive substrate inhibition therapy.

A rotating combination of two of the three therapeutic platforms (i.e. gene, enzyme and substrate inhibition therapy) may be used. However, subjects may also be treated by rotating (or overlapping) all three approaches as needed, as determined by the skilled clinician. Examples of treatment schedules may include but are not limited to: (1) gene therapy, then substrate inhibition followed by enzyme therapy; (2) enzyme therapy, then substrate inhibition followed by gene therapy; (3) gene therapy, then enzyme therapy followed by substrate inhibition therapy; (4) enzyme therapy, then gene therapy followed by substrate inhibition therapy. As noted previously, temporal overlap of therapeutic platforms may also be performed, as needed, depending on the clinical course of a given storage disease in a given subject.

A substrate inhibition component to a combination therapy is conceptually applicable to virtually all lysosomal storage disorders. LSDs amenable to treatment by substrate inhibition with DNJ and P4 type molecules include those of the glycosphingolipid pathway (e.g. Gaucher, Fabry, Tay-Sachs, Sandhoff and GM1-gangliosidosis).

The various macromolecules that accumulate in lysosomal storage diseases are not uniformly distributed, but instead are deposited in certain anatomic sites for each disease. However, an exogenously supplied enzyme, whether delivered by enzyme replacement therapy or gene therapy, is generally taken up by cells of the reticuloendothelial system and sorted to the lysosomal compartment where it acts to hydrolyze the accumulated substrate. Moreover, cellular uptake of therapeutic enzyme can be augmented by certain maneuvers to increase lysosomal targeting (see e.g. U.S. Pat. No. 5,549,892 by Friedman et al., assigned to Genzyme Corporation, which describes recombinant glucocerebrosidase having improved pharmacokinetics by virtue of remodeled oligosaccharide side chains recognized by cell surface mannose receptors which are endocytosed and transported to lysosomes).

Treatment intervals for various combination therapies can vary widely and may generally be different among different storage diseases and different individuals depending on how aggressively storage products are accumulated. For example, Fabry storage product accumulation may be slow compared to rapid storage product accumulation in Pompe. Titration of a particular storage disease in a particular individual is carried out by the skilled artisan by monitoring the clinical signs of disease progression and treatment success.

Some treatment modalities target some affected organs better than others. In Fabry, if ET does not reach the kidney well enough for a satisfactory clinical outcome, GT can be selectively targeted to the kidney (e.g. by injection). Other organs or disease loci such as bones and lung alveolar macrophages may not be well targeted by ET. Using GT, however, bones can be injected and lungs can be targeted with aerosols. SMT is able to cross the BBB, providing a powerful approach, when combined with GT and/or ERT, for treating LSDs having CNS manifestations. Moreover, substrate deprivation by SMT combined with enzyme replacement and/or gene therapy address the storage problem at separate and distinct intervention points which may enhance clinical outcome.

It will be understood that reference to simultaneous or concurrent administration of two or more therapies does not require that they be administered at the same time, just that they be acting in the subject at the same time.

M. Tissue Specific Promoters and Enhancers

In certain embodiments the invention provides an improved combination therapy for treating a subject with lysosomal storage disease, e.g., Fabry disease, Pompe disease, comprising administering to the subject a gene therapy vector encoding a lysosomal hydrolase, e.g., α-galactosidase A, α-glucosidase, where expression of the lysosomal hydrolase is controlled by at least one tissue specific regulatory element, e.g., a promoter, an enhancer, and administering at least one of a) an exogenously produced natural or recombinant lysosomal hydrolase; and b) a small molecule capable of treating a lysosomal storage disease. In some embodiments of the invention the gene therapy vector encoding a lysosomal hydrolase, is administered before the exogenously produced natural or recombinant lysosomal hydrolase or a small molecule capable of treating a lysosomal storage disease. In other embodiments the gene therapy vector encoding a lysosomal hydrolase, and at least one of the exogenously produced natural or recombinant lysosomal hydrolase and a small molecule capable of treating a lysosomal storage disease are administered contemporaneously. In yet other embodiments of the invention at least one of the exogenously produced natural or recombinant lysosomal hydrolase and a small molecule capable of treating a lysosomal storage disease are administered before the gene therapy vector encoding a lysosomal hydrolase.

Use of a gene therapy vector encoding a lysosomal hydrolase under the control of a tissue specific promoter may target expression of the lysosomal hydrolase to a specific tissue or organ, e.g., the liver, which may serve as a depot for production of the lysosomal hydrolase. Additionally use of a gene therapy vector encoding a lysosomal hydrolase under the control of a tissue specific promoter may increase the efficiency of infection, thus requiring administration of less vector, e.g. a viral vector to achieve a therapeutic effect. Tissue specific promoters may also permit selective up-regulation of the encoded lysosomal hydrolase in a tissue specific manner.

In certain embodiments, the tissue specific promoter is a liver specific promoter, e.g., an albumin promoter (see U.S. Patent Application No. 20030017139). In certain embodiments the gene therapy vector encoding a lysosomal hydrolase may further comprise at least one additional heterologous tissue specific regulatory element, e.g., enhancer. The tissue specific enhancer may be chosen from a human serum albumin enhancer, a human prothrombin enhancer, an α-1 microglobulin enhancer and an intronic aldolase enhancer.

Use of a gene therapy vector encoding a lysosomal hydrolase under the control of a liver specific promoter may reduce an immune response, e.g., a humoral response, to the lysosomal hydrolase encoded by the vector. Reducing the humoral response to the lysosomal hydrolase can increase both the half life and the concentration of the lysosomal hydrolase. This in turn may establish a basal level of expression of the lysosomal hydrolase. Establishing a basal level of expression of the lysosomal hydrolase allows for smaller, less frequent doses of subsequently administered exogenously produced natural or recombinant lysosomal hydrolase compared to, for example a treatment regimen involving a gene therapy vector without a tissue specific promoter or alternatively enzyme replacement therapy administered alone. Additionally, establishing a basal level of lysosomal hydrolase may also reduce the required small molecule dosage to treat the subject.

The use of a gene therapy vector encoding a lysosomal hydrolase under the contol of a liver specific promoter may also result in a state of immunological tolerance in the subject to the lysosomal hydrolase, thus overcoming a significant impediment in the treatment of lysosomal storage diseases. Initial induction of tolerance by first administering a gene therapy vector encoding a lysosomal hydrolase under the control of a tissue specific promoter, e.g., a liver specific promoter, may result in the need for subsequent treatments, e.g., enzyme replacement therapy, small molecule therapy, that are fewer and of shorter duration. Thus, unwanted side effects and complications associated with frequent and lengthy intravenous administration of exogenously produced natural or recombinant lysosomal hydrolase or administration of small molecule therapy may be avoided.

In one specific embodiment, the invention provides a method of treating Fabry disease comprising first administering a gene therapy vector encoding α-galactosidase A under the control of a human albumin promoter and two copies of a human prothrombin enhancer and followed by administration of:

(a) an exogenously produced natural or recombinant α-galactosidase A;

(b) a small molecule capable of treating Fabry disease, or

(c) both (a) and (b),

such that the Fabry disease is treated.

In another specific embodiment the invention provides a method of treating Pompe disease comprising first administering a gene therapy vector encoding α-glucosidase under the control of a liver specific promoter and optionally, at least one copy of a tissue specific enhancer and then administering at least one of the following:

a) an exogenously produced natural or recombinant α-glucosidase;

b) a small molecule capable of treating Pompe disease, or

(c) both (a) and (b),

such that the Pompe disease is treated.

The terms “treat,” “treatment,” “treating,” as used herein mean any of the following: reduction in severity of a disease or condition; reduction in the duration of a disease course; amelioration of one or more symptoms associated with a disease or condition; provision of beneficial effects to a subject with a disease or condition, without necessarily curing the disease or condition; or prophylaxis of one or more symptoms associated with a disease or condition.

### EXAMPLES

### Example 1

### Fabry Mice Treated with Enzyme Replacement and Small Molecule Therapy

Fabry mice were used to test the in vivo efficacy of combining enzyme replacement therapy with small molecule therapy in a sequential treatment format (FIG. 1). The study was designed to evaluate whether substrate inhibition (i.e. “substrate deprivation therapy”) using small molecules of the DNJ and P4 types could reduce re-accumulation of the storage material globotriaosylceramide (GL3). The study protocol (FIG. 1A) called for a single infusion of α-galactosidase A enzyme to reduce GL3 levels (measured at two weeks) to a “Baseline” level in Fabry mouse liver. GL3 re-accumulation was then measured at four weeks in control mice receiving no small molecule therapy (“Vehicle”) and in mice receiving various small molecules at various doses. Accordingly, two weeks after GL3 levels were reduced to a “Baseline” level of about 0.1 pg/g liver (FIG. 1B), a small molecule or vehicle was administered by intra-peritoneal (IP) injection. In the vehicle-treated control mice, GL3 re-accumulated to about 0.8 μg/gm liver tissue at the four week time point. By contrast, D-t-et-P4 (5 mg/kg) reduced GL3 re-accumulation to less than 0.4 μg/gm liver tissue at the four week time point. Similarly, AMP-DNJ (100 mg/kg) reduced GL3 re-accumulation to less than 0.3 μg/gm liver tissue at the four week time point. These results demonstrate the effectiveness of combination therapy in a storage disease mouse model. Specifically, small molecule therapy reduced the re-accumulation of storage material following its reduction by enzyme replacement therapy. These results also demonstrate the unexpected benefit of combining a hydrophobic DNJ derivative (i.e. AMP-DNJ) designed as a research tool for selective inhibition of a non-lysosomal enzyme (see (Overkleeft et al., 1998, J. Biol. Chem. 273, 26522-26527) with enzyme replacement.

### Example 2

### Construction and Production of Recombinant AAV2 Vectors Encoding Human α-galactosidase A

An AAV2 vector encoding human α-galactosidase A was constructed. The AAV2 vector plasmid used in the production of AAV2/CAAVHI-αgal was generated by subcloning the expression cassette encoding human α-galactosidase A into pNTC244 (Chejanovsky and Carter, 1989, Virology 171:239). The expression cassette is comprised of a human cytomegalovirus enhancer/promoter, a hybrid intron and bovine growth hormone polyadenylation signal sequence (Li et al., (2002) Mol. Ther. 5:731). A 1.2 kb fragment of the chicken 3-globin insulator was appended upstream of the expression cassette to increase the size of the vector to approximately 4.5 kb (Chung et al. 1997, Proc. Natl. Acad. Sci. USA 94: 575).

The plasmid vector used to produce AAV2/DC190-αgal was generated by subcloning the expression cassette encoding α-galactosidase A into pAAVSP70, which is a derivative of pAV1 (Laughlin et al. 1983, Gene 23:65). The expression cassette in AAV2/DC1 90-αgal is similar to that described for AAV2/CAAVHI-αgal except that the CAAV enhancer/promoter was replaced with the human serum albumin promoter (nucleotides −486 to +20) and to which were appended 2 copies of the human prothrombin enhancer (nucleotides −940 to −860). A 1.3 kb fragment of the human α,-antitrypsin 3′ intron (nucleotides 8110 to 9411) was also added upstream of the transcriptional cassette to increase the size of the vector to approximately 4.6 kb. Recombinant AAV2 vectors (Targeted Genetics Corporation, Seattle, Wash.) were generated by triple plasmid transfection of 293 cells and purified by column chromatography. Viral titers were determined using a real time TagMan® PCR assay using ABI PRISM 7700 (Applied Biosystems, Foster City, Calif.) with primers that were specific for the bovine growth hormone polyadenylation signal sequence.

### Example 3

### Administration of Viral Vectors and Exogenous Enzyme To Fabry and Balb/c Mice

Four to six week old male BALB/c mice were obtained from Taconic Laboratories (Germantown, N.Y.). Male Fabry −/− mice were bred at Genzyme Corporation (Framingham, Mass.) and allowed to mature to at least 4 months of age before use (Wang et al. 1996 Am. J. Hum. Genet. 59:A208).

The animals were cared for in an AAALAC accredited facility in accordance with the guidelines established by the National Research Council. For most of the studies, mice were administered 200 to 250 μl of the recombinant viral vectors or vehicle via the tail vein. Blood samples were collected from the orbital venous plexus under anesthesia (2-3% isoflurane) using heparinized microhematocrit capillary tubes. The animals were killed by injection with Euthasol (Delmarva Laboratories Inc., Midlothian, Va.) and their tissues then harvested and snap-frozen on dry ice. The samples were stored at −80° C. until ready for processing.

To suppress the formation of antibodies to α-galactosidase A in Fabry mice administered AAV2/CAAVHI-αgal, mice were treated with the anti-CD40 ligand monoclonal antibody MRt (BioExpress, Lebanon, N.H.). The dosage was 0.5 mg of the monoclonal antibody, in a total volume of 250 μl, injected intraperitoneally on day −1, 1, 3, 6, 9 and 13 relative to virus administration and this regimen was repeated every 4 weeks.

To test if mice that had been treated with AAV2/DC190-αgal had developed immune tolerance to α-galactosidase A, 50 μg of purified recombinant human a galactosidase A (Genzyme Corp., Boston, Mass.) in 100 μl phosphate-buffered saline were emulsified with 100 μl Complete Freund's adjuvant and injected intraperitoneally at 6 months post-treatment. Blood was collected 38 days later and assayed for the presence of anti-α-galactosidase A antibodies.

### Example 4

### Efficacy of Intravenous Administration of a Recombinant AAV2 Vector Encoding Human α-galactosidase A in Fabry Mice

To evaluate the relative utility of AAV vectors for treating Fabry disease, a recombinant AAV2 vector encoding human α-galactosidase A under the transcriptional control of the CAAV promoter (AAV2/CAAVHI-αgal) was constructed asa described above. Approximately 5×10¹¹ particles of AAV2/CAAVHI-αgal were then delivered via the tail vein to each immunosuppressed Fabry mouse. The mice were immunosuppressed because previous studies had shown that a robust humoral response against the expressed α-galactosidase A could be generated in these animals following gene transfer. The result was attenuated gene expression (see e.g. Ziegler et al. 1999, Hum. Gene Ther. 10:1667; Li et al. 2002, Mol. Ther. 5:731).

Tissue levels of α-galactosidase A were quantitated by an enzyme-linked immunosorbent assay (ELISA) using a polyclonal antibody to α-galactosidase A as described previously (Ziegler et al. 1989, Human Gene Therapy 10: 1667). This antibody recognizes human but not mouse α-galactosidase A. The method for extraction and purification of GL-3 from tissues was also essentially as described previously (Ziegler et al. 1989, Human Gene Therapy 10:1667). GL-3 levels were measured by an ELISA that was based on the ability of the E. coli verotoxin B subunit to bind the glycosphingolipid GL-3 (Ziegler et al. 1999, Anal. Biochem. 267:104).

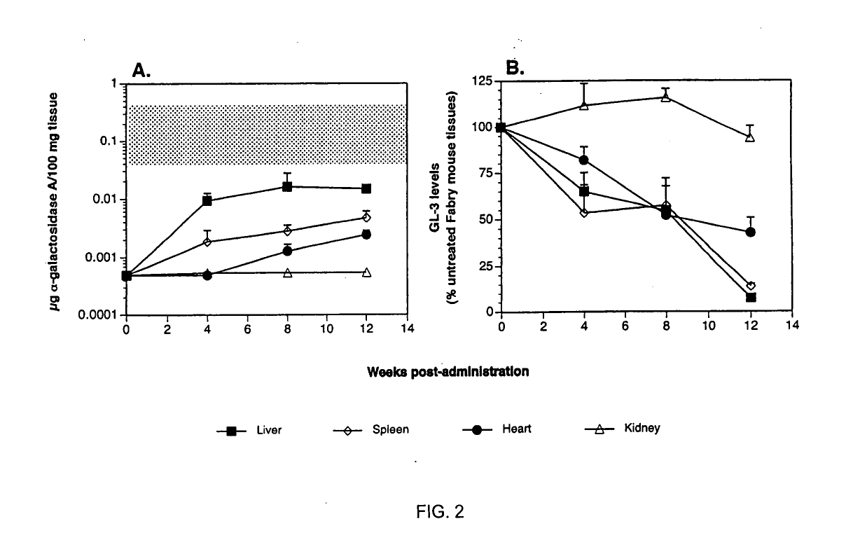

As expected, peak levels of the enzyme were detected in the livers of the treated animals at 4 weeks (FIG. 2A). The levels of α-galactosidase A attained in the livers were approximately 10% of those observed in normal mice and were sustained for the duration of the study (12 weeks). Lower amounts of the enzyme were also detected in the spleens and hearts but none was detected in the kidneys (FIG. 2A). These levels of α-galactosidase A attained with AAV2/CAAVHI-αgal were significantly lower (by 2 to 3 logs) than in animals administered an equivalent dose of a recombinant adenoviral vector containing the same transcriptional cassette (see Ziegler et al. 1999, Hum. Gene Ther. 10:1667; Ziegler et al. 2002, Hum. Gene Ther. 13:935).

Despite the relatively low levels of enzyme attained in the different organs with AAV2/CAAVHI-α-gal, they were sufficient to reduce the accumulated GL-3 content in these organs (FIG. 2B). The levels of GL-3 in the livers and spleens were reduced to basal levels and those in the heart were reduced by approximately 50% at 12 weeks post-treatment. However, no significant change in GL-3 levels was noted in the kidneys (FIG. 2B). The reductions in GL-3 corresponded with the enzyme levels attained in the different organs. Hence, sustained expression of relatively low amounts of α-galactosidase A was efficacious in clearing the GL-3 from some of the Fabry-affected tissues. The kinetics by which GL-3 was reduced in the tissues was relatively slow, with complete correction in the liver and spleen attained only after 12 weeks. This contrasts with results observed previously with adenoviral vectors encoding α-galactosidase A where complete clearance of GL-3 was achieved within 7 days of treatment (Ziegler et al. 1999, Hum. Gen. Ther. 10:1667).

### Example 5

### Enhanced and Sustained Expression of α-galactosidase A in Immuno-competent Mice Using the Liver-Specific Enhancer/Promoter, DC190

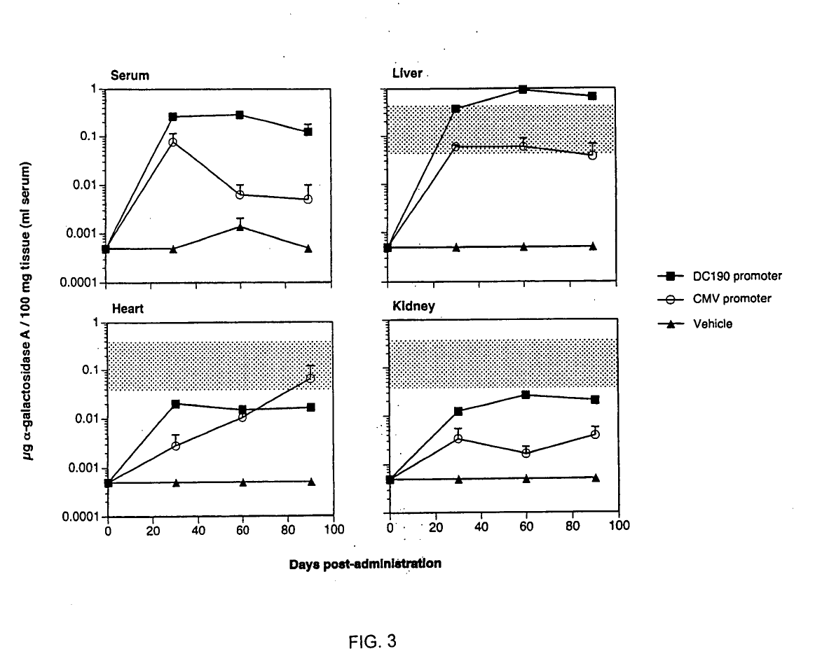

In an attempt to improve expression levels, several liver-specific enhancer/promoters capable of conferring high and persistent levels of expression of various transgenes in mice (Pastore et al. 1999, Hum. Gene Ther. 10:1773; Wang et al. 2000, Mol. Ther. 1:154) were evaluated. One such promoter (DC190), comprised of the human serum albumin promoter to which two copies of the human prothrombin enhancer were appended, demonstrated higher levels of expression than the CAAV promoter used in Example 4 (FIG. 3). Quantitiation of α-galactosidase A and GL-3 was as described in Example 4.

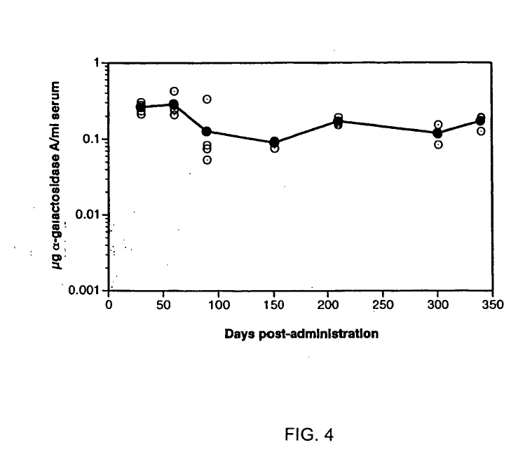

Intravenous administration of 3×1011 particles of AAV2/DC190-α-gal into BALB/c mice generated an approximately 15-fold greater level of α-galactosidase A than AAV2/CAAVHI-α-gal in the liver (FIG. 3). Consequently, correspondingly higher levels of the hydrolase were also detected in the serum, hearts and kidneys of these mice. Additionally, the expression levels in the serum of animals treated with AAV2/DC190-αgal were sustained for up to 340 days post-treatment (FIG. 4). Hence, use of the liver-restricted enhancer/promoter DC190 allowed for expression of higher levels of the lysosomal enzyme that were sustained for nearly a year in BALB/c mice.

### Example 6

### α-Galactosidase A Expression Directed by the DC190 Enhancer/Promoter

To examine and contrast the tissue specificity of expression of the liver-specific enhancer/promoter DC190 with that of the CAAV enhancer/promoter, BALB/c mice were administered 3×10¹¹ particles of either AAV2/DC190-αgal or AAV2/CAAVHI-αgal via the tail vein. The animals were killed 30 days later and their tissues harvested for analysis by real-time semi-quantitative TaqMan® PCR and quantitative RT-PCR.

To quantify AAV2 DNA tissue collection was performed using sterile, depurinated instruments. Genomic DNA was extracted with 1 ml of lysis buffer (100 mM Tris, 5 mM EDTA, 0.2% w/v SDS, 200 mM NaCl, and 30 mg Proteinase K, pH 8.5) for 18 hours at 60° C. The DNA was precipitated from the lysates with isopropyl alcohol and then washed with 75% (v/v) ethanol. After drying for 15 minutes, the pellets were resuspended in diethylpyrocarbonate treated water to a final DNA concentration of 100 ng/μl. AAV2 DNA was detected using a real-time semi-quantitative TaqMan® PCR assay using an ABI PRISM 7700 (Applied Biosystems, Foster City, Calif.) using a target sequence that was specific to the human α-galactosidase A cDNA. Five hundred ng of genomic DNA was amplified in each 50 μl reaction. Cycling conditions used were 50° C. for 2 minutes, 95° C. for 10 minutes, followed by 40 cycles at 95° C. for 15 seconds and 60° C. for 1 minute. The standard curve used in this assay was made from 10-fold serial dilutions of a plasmid DNA containing the target sequence (101 to 10⁵ copies) spiked into 500 ng of BALB/c mouse tissue genomic DNA. All samples in which amplification was not detected within 40 cycles, or where the copy number was calculated as less than 1 were reported as negative.

To quantify α-galactosidase A mRNA tissue samples were homogenized in 1 ml RNA-Stat 60 solution (Tel-Test B, Inc.; Friendswood, Tex.) and the RNA extracted according to the manufacturer's instructions. The extracted RNA was then resuspended in diethylpyrocarbonate-treated water to a final concentration of 1 ug/plI. Two pg aliquots were treated with DNase at 37° C. for 20 minutes after which they were then subjected to reverse transcription using a primer that was specific for human α-galactosidase A. Following completion of this reaction, the samples were amplified by TaqMan as described above

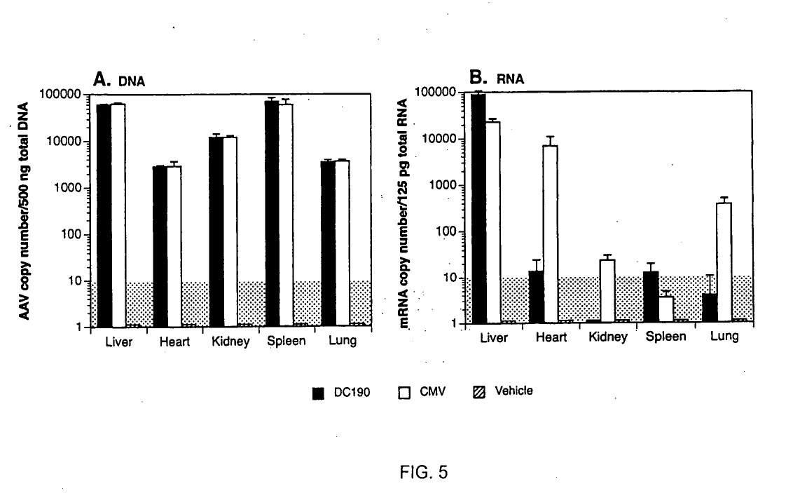

The tissue bio-distribution of both viral genomes was very similar (FIG. 5A). Highest levels of the viral DNAs were detected in the liver and spleen followed by the kidney and then the lung and heart. However, in contrast to animals administered AAV2/CAAVHI-αgal, where expression of α-galactosidase A mRNA was detected in the liver, heart, lung and to a lesser extent, the kidney, expression from the DC190 enhancer/promoter was largely restricted to the liver with minimal amounts detected in the heart and spleen (FIG. 5B). Hence, despite the delivery of the AAV2/DC190-αgal genome to a variety of tissues, expression of α-galactosidase A mRNA was primarily restricted to the liver.

### Example 7

### A Reduced Immune Response to α-Galactosidase A in BALB/c Mice Administered AAV2/DC190-αgal

The antibody response in BALB/c mice treated with either AAV2/DC190-αgal or AAV2/CAAVHI-αgal was investigated. The level of AAV2-specific antibodies in the serum was determined by ELISA. Serial dilutions of serum were added to wells of a 96-well plate coated with heat inactivated AAV2. Bound virus-specific antibodies were detected using horseradishperoxidase (HRP)-conjugated goat anti-mouse immunoglobulin G (IgG), IgM and IgA (Zymed, San Francisco, Calif.). Plates were incubated with SigmaFast OPD substrate (Sigma, St. Louis, Mo.) for 30 minutes for color development. Titers were defined as the reciprocal of the highest dilution of serum that produced an OD490 equal to or less than 0.1. Levels of α-galactosidase A-specific antibodies were similarly determined by ELISA except that the 96-well plates were coated with 1 pg/ml highly purified recombinant human α-galactosidase A.

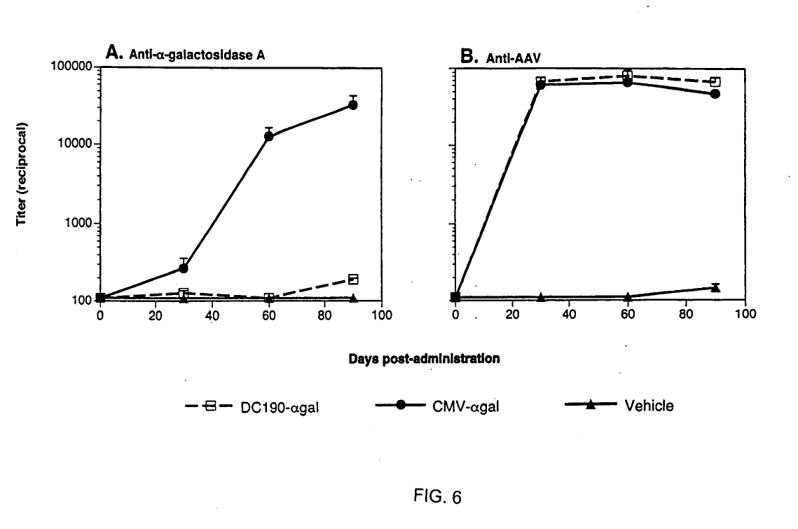

Mice administered AAV2/CAAVHI-αgal generated antibodies against α-galactosidase A starting at day 30 post-treatment (FIG. 6A) that remained elevated for a year (data not shown). The induction of high levels of these antibodies (day 60) coincided with the drop in expression of α-galctosidase A observed in these mice (FIG. 3). Interestingly, although mice administered AAV2/DC190-αgal generated higher levels of expression of the enzyme (FIG. 3), very low or no anti-α-galactosidase A antibodies were detected in these animals (FIG. 6A). Monitoring of AAV2/DC190-αgal treated mice beyond the 90 days showed no significant increase in their antibody titer to α-galactosidase A (data not shown). This reduced ability to mount an antibody response to α-galactosidase A in the AAV2/DC190-αgal-treated mice was not related to a loss or compromise of their immune competence as indicated by their ability to induce high titers of anti-AAV2 capsid antibodies (FIG. 6B). Hence in contrast to the CAAV promoter, use of the liver-restricted enhancer/promoter DC190 was associated with a reduced host immune response to the encoded transgene product. This lack of a robust immune response to α-galactosidase A in the AAV2/DC190-αgal-treated mice likely accounted for the high and sustained expression levels of the enzyme observed in these animals (FIGS. 3 and 4).

### Example 8

### Induction of Immune Tolerance to α-Galactosidase A in AAV2/DC190-αgal-Treated BALB/c Mice

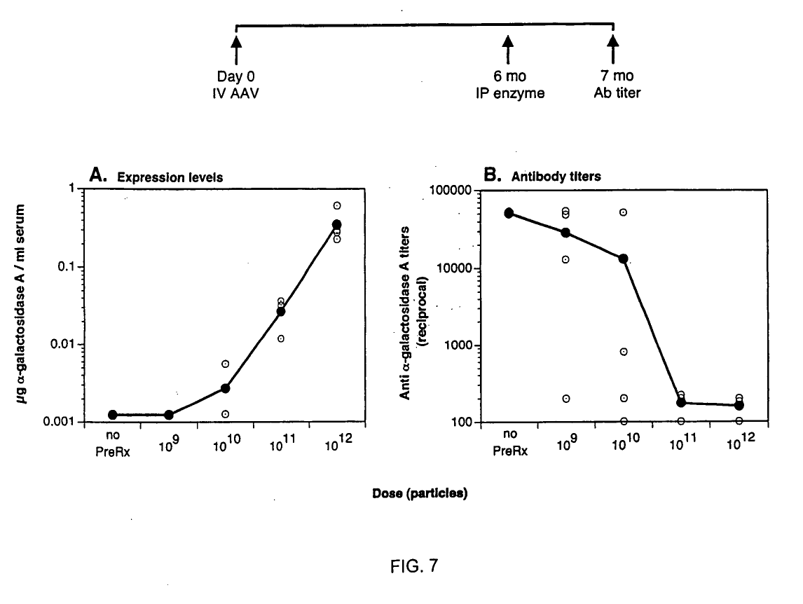

We next examined if the sustained expression observed in the AAV2/DC190 agal-treated BALB/c mice was associated with the induction of immune tolerance. Separate groups of mice were administered increasing amounts of AAV2/DC190-αgal and the expression levels of α-galactosidase A were monitored for 6 months. FIG. 7A shows that BALB/c mice treated intravenously with increasing amounts AAV2/DC190-αgal and assayed 88 days later exhibited correspondingly higher levels of the enzyme in the serum. Consistent with the earlier observations, these enzyme levels remained undiminished at 6 months post-treatment (data not shown) and there were no detectable anti-α-galactosidase A antibodies. After 6 months, the mice were challenged intraperitoneally with 50 pg of purified recombinant human α-galactosidase A emulsified in complete Freund's adjuvant. Serum was then collected 38 days later and assayed for the presence of anti-α-galactosidase A antibodies. Mice that had not been pre-treated with AAV2/DC190-αgal and then assayed 38 days later exhibited a robust antibody response to α-galactosidase A (FIG. 7B). In contrast, mice administered 10¹¹ or 10¹² particles of AAV2/DC190-αgal, which generated high levels of expression of the enzyme, did not elicit any measurable antibodies following the subsequent immunological challenge with purified enzyme. The lower dose cohorts generated mixed results with 3 out of 4 animals in the 10⁹ group and 2 out of the 4 animals in the 10¹⁰ group demonstrating measurable anti-α-galactosidase A antibodies (FIG. 7B). Hence, mice administered AAV2/DC190-agal have developed immune tolerance. This induction of immune tolerance was greater and more complete in mice that were administered higher doses of the recombinant AAV2 vector resulting in greater levels of expression of the enzyme.

### Example 9

### Systemic Administration of AAV21DC190-αgal Improved the Clearance of GL-3 in Fabry Mice

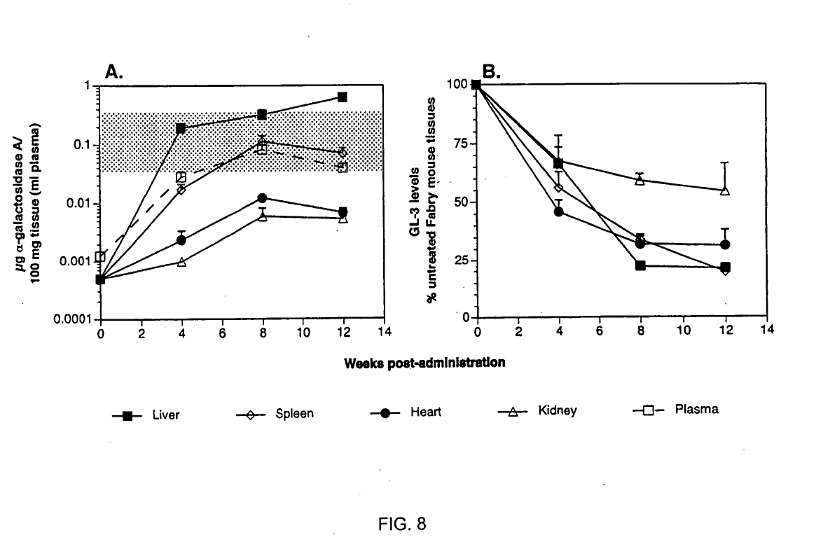

To determine if the improved characteristics shown associated with AAV2/DC190-αgal would provide greater efficacy in the Fabry mice, animals were intravenously administered 5×10¹¹ particles of the recombinant viral vector. FIG. 8A shows that AAV2/DC190-αgal-mediated gene transfer resulted in higher levels (approximately 20-fold) of expression of α-galactosidase A in the liver than were attained with AAV2/CAAVHI-αgal (FIG. 2A). Correspondingly greater levels of enzyme were also realized in the serum and other Fabry-affected visceral organs. Importantly, amounts of the hydrolase were also detected in the kidney, whereas none were measurable previously with AAV2/CAAVHI-αgal. Associated with these elevated levels of enzyme was an increase in the rate of GL-3 clearance from the tissues (FIG. 8B). Basal levels of GL-3 in the liver, heart and spleen were attained after 8 weeks instead of 12 weeks with AAV2/CAAVHI-αgal. Moreover, an approximately 40% reduction in substrate levels in the kidney was achieved using the improved transcriptional cassette. In contrast, no reduction was seen with the CAAV promoter. Moreover, unlike the earlier study with AAV2/CAAVHI-αgal that was performed with immunosuppressed Fabry mice, these improvements in overall enzyme levels and clearance of substrate with AAV2/DC190 αgal were accomplished in immunocompetent animals. Together, these data indicate that the use of the liver-restricted enhancer/promoter resulted in significantly improved efficacy in clearing GL-3 in the Fabry mice.

### Example 10

### A Combination Therapy for Treating Pompe Disease Using Gene and Enzyme Replacement Therapies

Preclinical research and early clinical studies have indicated that a relatively high dose of recombinant α-glucosidase (rhGAA) may be required to effectively treat the affected tissues of Pompe patients. For example, current trials in Pompe disease use doses that are in the range of ten milligrams, or more, of the enzyme. This is compared to 1-2 mg used for treating other lysosomal storage disorders. Additionally, the induction of antibodies to the infused enzyme presents an additional problem in treating subjects with Pompe disease.

One way to address these potential problems involves the use of combination enzyme replacement and gene therapies. Gene mediated expression of GM using AAV (adeno-associated virus) whereby the transcription of the enzyme is under the control of a liver restricted promoter can result in the induction of immune tolerance. Furthermore, AAV vectors, because of their ability to facilitate sustained, low level expression (greater than 1 year in mice), may provide for a lower dosing regimen with enzyme to treat Pompe-affected tissues.

The expression levels of GM will be assessed in an anaimal model, using AAV2/1 and AAV2/8-DC190GAA (encoding GAA). The antibody response to GM in Pompe mice will also be assessed.

Three mice per study group divided amongst three study groups (AAV2/1, AAV2/8, vehicle) will be used. The study will run 28 days with serum samples taken 1 week, 2 weeks, 4 weeks post administration. Serum will be assayed for GM enzyme activity and anti-GM antibody titers. Numerous tissue samples (e.g., liver, heart, and several different muscle groups) will be taken at sacrifice on day 28 to assess GM and glycogen levels.

### Example 11

### Induction of Immune Tolerance in Pompe Disease

The level of GM expression necessary to achieve immune tolerance in Pompe mice will be determined. The most effective serotype vector (AAV2/1 or AAV2/8) capable of conferring immune tolerance will also be determined. Lastly, the time necessary following vector administration for development of immune tolerance will be determined.

Three doses of an AAV vector encoding α-glucosidase (3×10⁹, 3×10¹⁰, 3×10¹¹ particles) will be administered to mice to generate various levels of GM expression. At each vector dose a group of 4 mice will be challenged with rhGM protein at (minimally) two time points. The first time point will be determined by an ongoing tolerization study in Fabry mice to determine the minimal time required to tolerate challenge with α-galactosidase A. The second time point will be scheduled at a later date in case a longer time span is required to tolerize mice to GM compared to α-galactosidase A. Serum samples will be taken at regular time intervals throughout the study. Serum will be assayed for GM enzyme activity and anti-GM antibody titers. Numerous tissue samples (liver, heart, and several different muscle groups) will be taken at sacrifice to assess GM enzyme activity and glycogen levels.

### Example 12

### The Effect of Low Dose Gene Therapy on Enzyme Replacement Therapy Levels Required to Clear Substrate

A low dose of AAV vector (sufficient to tolerize, but not to clear substrate) (3×10⁸-3×10¹⁰ particles) will be administered to mice to determine if this allows for a lower dose of enzyme to clear substrate from affected tissues (heart, diaphram, skeletal muscle).

The following treatment groups will be established:

1) 100 mg/kg rhGAA (shown to be efficacious in previous studies)

2) 20 mg/kg rhGAA (shown to be insufficient in previous studies)

3) AAV & 20 mg/kg rhGAA

4) AAV alone

5) Vehicle alone

Groups of 4 Pompe mice will be pre-treated with gene therapy or vehicle prior to enzyme replacement therapy (timing to be determined by Example 15). Four weekly doses of rhGAA will then be administered, and animals will be sacrificed 7 days following the last enzyme administration. Serum samples will be taken at regular time intervals throughout. Serum will be assayed for GAA enzyme activity and anti-GAA antibody titers. Numerous tissue samples (e.g., liver, heart, and several different muscle groups) will be taken at sacrifice to assess GAA enzyme activity and glycogen levels.

All references cited herein are incorporated herein by reference in their entirety and for all purposes to the same extent as if each individual publication or patent or patent application was specifically and individually indicated to be incorporated by reference in its entirety for all purposes. To the extent publications and patents or patent applications incorporated by reference contradict the disclosure contained in the specification, the specification is intended to supercede and/or take precedence over any such contradictory material.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Many modifications and variations of this invention can be made without departing from its spirit and scope, as will be apparent to those skilled in the art. The specific embodiments described herein are offered by way of example only and are not meant to be limiting in any way. It is intended that the specification and examples be considered as exemplary only, with the true scope and spirit of the invention being indicated by the following claims.

What is claimed is:

1. A method of treating a subject diagnosed as having a lysosomal storage disease comprising administering a gene therapy vector encoding a lysosomal hydrolase under the control of at least one tissue specific regulatory element and administering:
(a) an exogenously produced natural or recombinant lysosomal hydrolase;
(b) a small molecule capable of treating a lysosomal storage disease, or
(c) both (a) and (b),
such that the lysosomal storage disease is treated.

2. The method of claim 1, where the gene therapy vector encoding a lysosomal hydrolase under the control of a tissue specific regulatory element is administered before the exogenously produced natural or recombinant lysosomal hydrolase or the small molecule capable of treating a lysosomal storage disease.

3. The method of claim 1, where the tissue specific regulatory element is chosen from at least one of a tissue specific promoter and a tissue specific enhancer.

4. The method of claim 1, where administering the gene therapy vector encoding a lysosomal hydrolase induces immunological tolerance to the lysosomal hydrolase.

5. The method of claim 1, where administration of the gene therapy vector encoding a lysosomal hydrolase under the control of a tissue specific promoter is followed by administration of an exogenously produced natural or recombinant lysosomal hydrolase.

6. The method of claim 5, where the amount of the exogenously produced natural or recombinant lysosomal hydrolase administered to the subject is less than the amount administered to treat a subject with a lysosomal storage disease that has not been administered a gene therapy vector encoding a lysosomal hydrolase or has been administered a gene therapy vector without a tissue specific promoter controlling expression of the lysosomal hydrolase.

7. The method of claim 1, where the lysosomal storage disease is Fabry disease.

8. The method of claim 7, where the treatment results in a decrease in GL-3 in the subject compared to the GL-3 level in the subject before treatment.

9. The method of claim 7, where the lysosomal hydrolase is α-galactosidase A.

10. The method of claim 1, where the lysosomal storage disease is Pompe disease.

11. The method of claim 10, where the treatment results in a decrease in glycogen in the subject compared to the glycogen level in the subject before treatment.

12. The method of claim 10, where the lysosomal hydrolase is α-glucosidase.

13. The method of claim 1, where the gene therapy vector is a viral vector.

14. The method of claim 11, where the viral vector is chosen from AAV1, AAV2, AAV5, AAV7 and AAV8.

15. The method of claim 1, where the tissue specific regulatory element is a liver specific promoter.

16. The method of claim 15, where the liver specific promoter is a human serum albumin promoter.

17. The method of claim 1, where tissue specific regulatory element is a tissue specific enhancer.

18. The method of claim 17, where the tissue specific enhancer is a human prothrombin enhancer.

19. The method of claim 1, where the small molecule capable of treating a lysosomal storage disease is chosen from deoxynojirimycin, N-propyldeoxynojirimycin, N-butyideoxynojirimycin, N-butyldeoxygalactonojirimycin, N-pentlydeoxynojirimycin, N-heptyldeoxynojirimycin, N-pentanoyldeoxynojirimycin, N-(5-adamantane-1-ylmethoxy)pentyl)-deoxynojirimycin, N-(5-cholesteroxypentyl)-deoxynojirimycin, N-(4-adamantanemethanylcarboxy-1-oxo)-deoxynojirimycin, N-(4-adamantanylcarboxy-1-oxo)-deoxynojirimycin, N-(4-phenantrylcarboxy-1-oxo)-deoxynojirimycin, N-(4-cholesterylcarboxy-1-oxo)-deoxynojirimycin, or N-(4-b-cholestanylcarboxy-1-oxo)-deoxynojirimycin, D-threo-1-phenyl-2-palm itoylamino-3-pyrrolidino-1-propanol (P4), D-threo-4′-hydroxy-1-phenyl-2-palmitoylamino-3-pyrrolidino-1-propanol (4′-hydroxy-P4), D-threo-1-(3′,4′-trimethylenedioxy)phenyl-2-palmitoylamino-3-pyrrolidino-1-propanol (trimethylenedioxy-P4), D-threo-1-(3′,4′-methylenedioxy)phenyl-2-palmitoylamino-3-pyrrolidino-1-propanol (methylenedioxy-P4) and D-threo-1-(3′,4′-ethylenedioxy)phenyl-2-palmitoylamino-3-pyrrolidino-1-propanol (ethylenedioxy-P4 or D-t-et-P4).

20. A method of treating a subject diagnosed as having Fabry disease comprising administering a gene therapy vector encoding α-galactosidase A under the control of a human albumin promoter and 2 copies of a human prothrombin enhancer and administering:
(a) an exogenously produced natural or recombinant α-galactosidase A;
(b) a small molecule capable of treating Fabry disease, or
(c) both (a) and (b),
such that the Fabry disease is treated.

21. The method of claim 20, where the gene therapy vector encoding α-galactosidase A under the control of a human albumin promoter and 2 copies of a human prothrombin enhancer is administered before the exogenously produced natural or recombinant α-galactosidase A or a small molecule capable of treating Fabry disease.

22. A method of treating a subject diagnosed as having Pompe disease comprising first administering a gene therapy vector encoding α-glucosidase under the control of a liver specific promoter and optionally, at least one copy of a tissue specific enhancer followed by administration of:
(a) an exogenously produced natural or recombinant α-glucosidase;
(b) a small molecule capable of treating Pompe disease, or
(c) both (a) and (b),
such that the Pompe disease is treated.

23. A composition useful for treating a lysosomal storage disease comprising a gene therapy vector encoding a lysosomal hydrolase under the control of a tissue specific regulatory element and (a) an exogenously produced natural or recombinant lysosomal hydrolase; (b) a small molecule capable of treating a lysosomal storage disease or (c) both (a) and (b).

24. The composition of claim 23, where the gene therapy vector encoding a lysosomal hydrolase encodes α-galactosidase A.

25. The composition of claim 23, where the gene therapy vector encoding a lysosomal hydrolase encodes α-glucosidase.

26. The composition of claim 23, where the gene therapy vector is a viral vector.

27. The composition of claim 26, where the viral vector is chosen from AAV1, AAV2, AAV5, AAV7 and AAV8.

28. The composition of claim 23, where the exogenously produced natural or recombinant lysosomal hydrolase is chosen from α-galactosidase A and α-glucosidase.

29. The composition of claim 23, where the tissue specific regulatory element is a liver specific promoter.

30. The composition of claim 29, where the liver specific promoter is an albumin promoter.

31. The composition of claim 23, where the tissue specific regulatory element is a tissue specific enhancer.

32. The composition of claim 31, where the tissue specific enhancer is a human prothrombin enhancer.

33. The composition of claim 23, where the small molecule capable of treating a lysosomal storage disease is chosen from deoxynojirimycin, N-propyldeoxynojirimycin, N-butyideoxynojirimycin, N-butyideoxygalactonojirimycin, N-pentlydeoxynojirimycin, N-heptyldeoxynojirimycin, N-pentanoyideoxynojirimycin, N-(5-adamantane-1-ylmethoxy)pentyl)-deoxynojirimycin, N-(5-cholesteroxypentyl)-deoxynojirimycin, N-(4-adamantanemethanylcarboxy-1-oxo)-deoxynojirimycin, N-(4-adamantanylcarboxy-1-oxo)-deoxynojirimycin, N-(4-phenantrylcarboxy-1-oxo)-deoxynojirimycin, N-(4-cholesterylcarboxy-1-oxo)-deoxynojirimycin, or N-(4-b-cholestanylcarboxy-1-oxo)-deoxynojirimycin, D-threo-1-phenyl-2-palmitoylamino-3-pyrrolidino-1-propanol (P4), D-threo-4′-hydroxy-1-phenyl-2-palmitoylamino-3-pyrrolidino-1-propanol (4′-hydroxy-P4), D-threo-1-(3′,4′-trimethylenedioxy)phenyl-2-palmitoylamino-3-pyrrolidino-1-propanol (trimethylenedioxy-P4), D-threo-1-(3′,4′-methylenedioxy)phenyl-2-palmitoylamino-3-pyrrolidino-1-propanol (methylenedioxy-P4) and D-threo-1-(3′,4′-ethylenedioxy)phenyl-2-palmitoylamino-3-pyrrolidino-1-propanol (ethylenedioxy-P4 or D-t-et-P4).

34. A composition useful for treating Fabry disease comprising a gene therapy vector encoding α-galactosidase A under the control of a human albumin promoter and 2 copies of a human prothrombin enhancer and:
(a) an exogenously produced natural or recombinant α-galactosidase A;
(b) a small molecule capable of treating Fabry disease, or
(c) both (a) and (b).

35. A composition useful for treating Pompe disease comprising a gene therapy vector encoding α-glucosidase under the control of a liver specific promoter and optionally at least one tissue specific enhancer and:
a) an exogenously produced natural or recombinant α-glucosidase;
b) a small molecule capable of treating Pompe disease or
(c) both (a) and (b).
